(19) United States
(12) Patent Application Publication
(10) Pub. No.: US 2018/0073062 A1
(43) Pub. Date: Mar. 15, 2018

(54) COMPOSITIONS AND METHODS FOR IDENTIFYING ENDOGENOUS DNA-DNA INTERACTIONS

(71) Applicant: The University of Chicago, Chicago, IL (US)

(72) Inventors: Geoffrey GREENE, Chicago, IL (US); Ryan BOURGO, Chicago, IL (US)

(21) Appl. No.: 15/706,277

(22) Filed: Sep. 15, 2017

Related U.S. Application Data
(60) Provisional application No. 62/395,130, filed on Sep. 15, 2016.

(51) Int. Cl. C12Q 1/68; C40B 30/04

(52) U.S. Cl. CPC C12Q 1/6832; C12Q 1/6876; C12Q 1/6806; C12Q 2565/518; C12Q 1/6816; C40B 30/04; C12Q 2565/519; C12Q 1/6811

(57) ABSTRACT

Provided herein are compositions and methods for identifying endogenous DNA-DNA interactions. In particular, compositions and methods are provided for performing Capture of Associated Targets on CHromatin (CATCH) assays which use efficient capture and enrichment of specific genomic loci of interest through hybridization and subsequent purification via complementary oligonucleotides, without the need for enzymatic digestion or ligation steps.

### CROSS-REFERENCE TO RELATED APPLICATIONS

The present invention claims priority to U.S. Provisional Patent Application 62/395,130, filed Sep. 15, 2016, which is incorporated by reference in its entirety.

### STATEMENT REGARDING FEDERAL FUNDING

This invention was made with government support under Grant Number(s) R01 CA089489 awarded by The National Institutes of Health. The government has certain rights in the invention.

### FIELD

Provided herein are compositions and methods for identifying endogenous DNA-DNA interactions. In particular, compositions and methods are provided for performing Capture of Associated Targets on CHromatin (CATCH) assays which use efficient capture and enrichment of specific genomic loci of interest through hybridization and subsequent purification via complementary oligonucleotides, without the need for enzymatic digestion or ligation steps.

### BACKGROUND

Chromatin architecture is a key regulator of many aspects of cell biology, including gene transcription, DNA repair processes, DNA replication, and long-term processes such as X-chromosome inactivation (ref 1; incorporated by reference in its entirety). A host of transcription factors, enzymes, scaffolding proteins, and other factors ensure that local chromatin architecture is a dynamic environment that directly, and indirectly regulates the complex cellular processes noted above (ref. 2; incorporated by reference in its entirety). This complex network of chromatin architecture is largely composed of the non-protein-coding genome. Estimates of the true functional percentage of our genome range from 10% to as much as 80% (refs. 3-4; incorporated by reference in their entireties), but despite this discrepancy, only a small fraction of the genome has been evolutionarily conserved, largely in those protein-coding regions. Transcriptional enhancers are disproportionately common amongst evolutionarily conserved non-protein coding sequences (ref. 5; incorporated by reference in its entirety).

Transcriptional enhancer regions are hubs of transcription factor binding, and are thought to underlie a significant portion of the tissue-specific expression of many gene targets (refs. 5-7; incorporated by reference in their entireties). Whereas gene promoters are enriched for H3K4me3, these enhancer regions contain almost exclusively the mono-methylated version of H3K4 (H3K4me1). In addition it has been found that most active enhancers are characterized by the increased presence of H3K27ac. The epigenetic enhancer signature (ref 8; incorporated by reference in its entirety) has greatly contributed to the genome-wide prediction of transcriptional enhancer sites. It has become increasingly clear that the majority of transcriptional enhancers are not located within or directly adjacent to the genes they modulate, but are typically located at great linear distance. Despite the long-range linear distance (in base pairs) between two interacting loci—in many cases, hundreds or thousands of kilobases—the prevailing model is that of a physical interaction between the two sites. This requires long-distance genomic looping to occur, whereby two linearly distant genomic loci come into close proximity, and are held together by complexes of proteins and transcription factors (ref 9; incorporated by reference in its entirety). While the looping mechanism of DNA-DNA interaction has been postulated for nearly four decades (ref 10; incorporated by reference in its entirety), it has been notoriously difficult to study.

Existing assays rely on random end-ligation at very low DNA concentrations after restriction digestion, which reduces assay reproducibility, and results in significant data loss (ref. 12; incorporated by reference in its entirety). These assays are easy to corrupt, difficult to troubleshoot, and are impractical to the average research laboratory (ref. 13; incorporated by reference in its entirety). Thus, the field of chromatin interaction is lacking in tools to facilitate mechanistic understanding of this important process.

### SUMMARY

Provided herein are compositions and methods for identifying endogenous DNA-DNA interactions. In particular, compositions and methods are provided for performing Capture of Associated Targets on CHromatin (CATCH) assays which use efficient capture and enrichment of specific genomic loci of interest through hybridization and subsequent purification via complementary oligonucleotides, without the need for enzymatic digestion or ligation steps.

Experiments conducted to develop a protocol (referred to herein as “Capture of Associated Targets on Chromatin” (“CATCH”)) that would overcome the current technological limitations (e.g., enzymatic digestion, ligation, etc.) that constrain the field of DNA-DNA interaction research. CATCH utilizes chemical crosslinking to capture naturally occurring nucleic acid-protein interactions, an unbiased sonication approach to shear DNA, enrichment of a genomic locus of interest through hybridization, and purification using a complementary labeled (e.g., biotinylated) oligonucleotide. Due to the crosslinking (e.g., formaldehyde crosslinking), this procedure purifies both the targeted DNA sequence and any interacting nucleic acid segments. Following de-crosslinking, the resulting DNA sample is subjected to analysis (e.g., PCR, sequencing, etc.) to identify interacting fragments.

Experiments were conducted during development of embodiments herein to demonstrate the effectiveness of CATCH by interrogating a downstream enhancer of the human SIAH2 gene, which had been previously analyzed using ChIA-PET. SIAH2 (3q25.1) is an E3 ubiquitin ligase whose up-regulation correlates with ER activation and has been linked to poor outcome in breast cancer patients (refs. 14-15; incorporated by reference in their entireties). Currently, SIAH2 gene transcriptional control is poorly understood: the only confirmed genomic loop within SIAH2 occurs between an intronic estrogen response element (ERE) and downstream ERE (ref. 16; incorporated by reference in its entirety), however, multiple ER binding sites are present within and around the gene. In order to resolve the interactions involved in SIAH2 regulation, as well as to demonstrate the looping events around SIAH2, next-generation sequencing was performed after CATCH of the SIAH2 downstream enhancer. In addition, experiments conducted during development of embodiments herein demonstrate the reproducibility of CATCH using distinct pull-downs near the SIAH2, EIF4A1, and MYC genes. These experiments also show that CATCH-seq peaks are overwhelmingly found overlapping with enhancers (H3K4me1 and H3K27ac enriched) and estrogen receptor (ER) binding sites. Finally, these experiments reveal unique subsets of physically interacting gene promoters that are shown to be transcriptionally co-expressed over thousands of data sets using the SEEK search system (ref. 17; incorporated by reference in its entirety).

In some embodiments, provided herein are methods comprising one or more (e.g., all) of the steps of: (a) fixing (e.g., crosslinking nucleic acids and/or protein within) a cell population to capture nucleic acid-protein-nucleic acid interactions; (b) sonicating the cell population to shear the nucleic acid into small fragments; (c) hybridizing nucleic acid target sequences to a labeled oligo; (d) separating the hybridized nucleic acid from unhybridized nucleic acid, thereby enriching for target sequences and associated protein-nucleic acid complexes; (e) de-crosslinking; and (f) analyzing target sequences and any associated nucleic acid sequences. In some embodiments, the cell population is formaldehyde fixed. In some embodiments, the labeled oligo is a biotinylated oligo. In some embodiments, the hybridized nucleic acid is separated from the unhybridized nucleic acid using streptavidin-linked magnetic beads. In some embodiments, analyzing target sequences and any associated nucleic acid sequences comprises performing PCR amplification. In some embodiments, analyzing target sequences and any associated nucleic acid sequences comprises next-generation sequencing. In some embodiments, the method results in the identification of DNA sequences physically associated with the target acid target sequences (via proteins). In some embodiments, the method does not comprise an enzymatic digestion step. In some embodiments, the method does not comprise a ligation step. In some embodiments, the target nucleic acid and/or associated nucleic acid is DNA. In some embodiments, the target nucleic acid and/or associated nucleic acid is RNA.

In some embodiments, provided herein are compositions, systems, or kits for performing the methods described herein (e.g., labelled oligonucleotides, fixing reagents, amplification reagents, sequencing reagents, tags and corresponding capture reagents, etc.).

### BRIEF DESCRIPTION OF THE DRAWINGS

FIG. 1. Exemplary process of Capture of Associated Targets on Chromatin. The flowchart demonstrates a basis of Capture of Associated Targets on Chromatin (CATCH). It shows that a cell population is first formaldehyde fixed to capture DNA-protein-DNA interaction. The DNA is then sheared into small fragments using sonication. The resulting fragmented DNA-protein-DNA complexes are hybridized to a biotinylated oligo in order to enrich for a region of interest. While the targeted sequence is pulled out of the entire DNA population using streptavidin-linked magnetic beads, any associated protein-DNA complexes are also enriched. Subsequent de-crosslinking and PCR amplify the target sequence and any (potentially) associated sequences. Next-generation sequencing allows the identification of any DNA sequences physically associated with the locus (via proteins) to which the biotinylated probe was originally hybridized.

FIG. 2. CATCH recapitulates chromatin interactions detected with ERα ChIA-PET. Top Diagram: The diagram represents the linear distances between (A) the downstream enhancer of SIAH2, (B) a SIAH2 intronic ERE, and (C-E) three additional sites shown, using ChIA-PET, to have interaction with site A in MCF-7 cells. Y-axis labels below IGV histograms represent number of sequencing reads after background subtraction. Sub-Panels: (A) The CATCH-seq pull-down region, also an enhancer downstream of SIAH2, is demonstrated to be highly enriched after CATCH-seq, confirming the efficacy of the capture method. (B) The SIAH2 intronic ERE, as denoted by the presence of ERα binding sites detected by ten different studies (gray bars, data sets outlined in methods section), was positive for two different interactions flanking the known ERα binding regions. (C) The downstream ERE shows interaction with an ERα binding region upstream of SIAH2; the location of the interaction was about 2 kb from the ERα binding site according to CATCH-seq. (D) an ERα binding site near LINC01213 and (E) a region of ERα binding within ARHGEF26 both show interaction with the SIAH2 downstream ERE.

FIG. 3. CATCH-seq demonstrates specificity and reproducibility. Y-axis labels below IGV histograms represent number of sequencing reads after background subtraction. (A) The promoter region of EIF4A1 was directly captured via CATCH, along with the promoter region of GRB7, which served as a negative control for DNA-DNA interaction. (B) Examples of raw CATCH-seq histograms (background subtracted) demonstrating the locations of peaks near gene promoters, and the reproducibility of multiple replicates. Histograms are on the same scale. (C) EIF4A1 promoter CATCH-seq interactions significantly overlap with enhancer marks (H3K4me1, H3K27ac) compared to control (greyscale), whose overlap is no greater than statistically random; the frequency of these overlaps increase with peak height. (D) The CATCH-seq interactions of the EIF4A1 promoter occur, on average, between 50-100 bp downstream of TSS's on chromosome 17. There was no discernable pattern near TSS's for the control pulldown. Both density plots have identical X and Y axes. (E) IPA analysis detailing specific gene promoters that demonstrated physical interaction with the EIF4A1 promoter. There was a remarkable 72% overlap between the CATCH-seq replicates.

FIG. 4. CATCH-seq of enhancer downstream of SIAH2 reveals plasticity of genomic architecture. Y-axis labels below IGV histograms represent number of sequencing reads after background subtraction. (A) The captured region is the downstream enhancer of SIAH2 (T47D human breast cancer cells). Interaction peaks can be seen at the intronic enhancer and SIAH2 promoter in both the presence and absence of estradiol. (B) The CATCH-seq signal density within 2 kb of TSS's on chromosome 3 peaks at around +200 bp in both the presence and absence of estradiol. There was no discernable peak in the randomized dataset. (C) To-scale Venn diagram demonstrating the number of genes' promoters identified as interacting with the downstream enhancer of SIAH2. The majority of genes are unchanged with the addition of estradiol (264); however, estradiol treatment does induce the loss of interaction (107) and the gain of interaction (140) of a large subset of genes. (D) Examples of raw CATCH-seq histograms demonstrating the striking overlap between ERα binding sites, enhancer marks, and CATCH-seq peaks near the promoters of genes. Many of the peaks (which indicate physical interaction with the downstream enhancer of SIAH2) are reduced/not found in the absence of estradiol, or enhanced upon the addition of estradiol. (E, left) SIAH2 enhancer CATCH-seq interaction peaks significantly overlap with enhancer marks and ER-binding sites compared to statistically random control; the frequency of these overlaps increase with peak height. (E, right) Increasing peak height thresholds reduce the number of CATCH-seq peaks identified at gene promoters, however at all thresholds, estradiol treatment facilitates more DNA-DNA interactions than vehicle control. (F) IPA analysis of two statistically significant processes identified under vehicle- (cell motility) and estradiol-treated (RNA expression) conditions. There is little overlap between the two processes.

FIG. 5. Finding CATCH-seq interactions at gene promoters predicts transcriptional co-expression. (A) Graphic outlining the general processing and data analysis that assess the ability of CATCH interactions to predict SEEK co-expressed genes. (B) Histogram representing the number of SEEK genes ‘predicted’ at random, or by specific CATCH-seq (left, gray: GRB7 negative control; right, green: EIF4A1 promoter). (C) Histogram representing the number of SEEK genes ‘predicted’ at random, or by specific CATCH-seq (orange: MYC downstream enhancer). (D) Histogram representing the number of SEEK genes ‘predicted’ at random, or by specific CATCH-seq (left, light blue: SIAH2-vehicle; right, dark blue: SIAH2-estradiol). In each experimental case, CATCH was capable of significantly predicting SEEK co-expression over random. Analyses were restricted to the specific chromosome of the CATCH pull-down. P-values were calculated via t-distribution.

FIG. 6. (A) This diagram represents the relative location of several key EREs within and around the human SIAH2 gene that were tested via CATCH in part B. (B) The DNA gel image shows that when specifically capturing the EREB locus within the intron of SIAH2, incomplete DNA sonication allows capture of a locus approximately 1.1 kb away (EREA). In addition, lack of CH2O fixation results in capture of only the targeted fragment. However, with complete sonication and fixation, the ERE downstream of SIAH2 is also captured with the EREB (DNSTRM). (C) This is a DNA gel image showing representative genomic DNA fragment size after complete sonication, using both unfixed (native) and fixed (1% CH2O) MCF-7 cells.

FIG. 7. These histograms (same scale, Y-axis) show that CATCH-seq requires 2nd strand synthesis to visualize sequencing from the captured region (complimentary to the biotinylated oligo). The presence of the biotinylated oligo during the CATCH protocol prevents that portion of the genome from rehybridizing with its natural complimentary region of DNA. Subsequently, without second strand synthesis, this locus cannot be detected by sequencing.

FIG. 8. This is a chromatin immunoprecipitation experiment showing ERα binding at various locations around the SIAH2 gene before and after E2 treatment. This experiment validated previous work showing ERα binding at the intronic and downstream EREs, as well as showing a lack of ERα binding at the SIAH2 promoter in MCF-7 cells, which corroborates previous findings.

FIG. 9. (A) Human TFF1 is known to have an ERα-binding enhancer region approximately 9.5 kb upstream of its TSS. This diagram shows the approximate location of that enhancer (red circle), the promoter region (blue circle), and each “random” site (grey circles) tested via CATCH, relative to each other and relative to the TFF1 gene (blue line). (B) ChIP experiment showing the responsiveness of the TFF1 promoter to E2 treatment at 45 minutes. Served to validate previous findings, as well as positive control for E2-responiveness in FIG. 8. (C) The gel image shows the relative intensity of PCR products associated with each tested locus around TFF1. The gDNA gel represents the total genomic input to the CATCH protocol, while the ENHCR capture gel represents just the pool of DNA that was enriched after CATCH was done targeting the enhancer region upstream of TFF1. The graph displays the mean (SEM error bars) of three experimental replicates. Student's unpaired t-test of data sets with unequal variance was performed to compare the strongest negative control signal against the promoter region (* denotes p<0.05).

FIG. 10. (A) Human PDZK1 contains both ER and PR binding sites just upstream of the TSS. This diagram shows the approximate location of both of those sites, as well as each “random” site (circles) tested via CATCH in part B. (B) The gel image shows the relative intensity of PCR products associated with each tested locus around PDZK1. The gDNA gel represents total genomic input, and the ERE capture visualizes only the pool of DNA that was enriched after CATCH targeting the estrogen binding element just upstream of the PDZK1 TSS. The graph displays the mean (SEM error bars) of three experimental replicates. Student's unpaired t43 test of data sets with unequal variance was performed to compare the strongest negative control signal against that of the PRE (* denotes p<0.05). (C) The human FHL2 gene has an ERE located about 5 kb upstream, and a PRE about 21 kb downstream within its second intron. This diagram shows the relative position of the ERE, PRE, and random sites tested in part D. (D) The gel image shows the relative intensity of PCR products associated with each tested locus around FHL2. The gDNA gel represents total genomic input, and the ERE capture visualizes only the pool of DNA that was enriched after CATCH targeting the estrogen binding element upstream of the FHL2 TSS. The graph displays the mean (SEM error bars) of three experimental replicates. Student's unpaired t-test of data sets with unequal variance was performed to compare the strongest negative control signal against that of the PRE (* denotes p<0.05).

FIG. 11. (A) An enhancer region downstream of MYC was directly captured via CATCH. (B) Enhancer downstream of MYC CATCH-seq interactions significantly overlap with enhancer marks compared to statistically randomized control; the frequency of these overlaps increase with peak height. (C) The CATCH-seq interactions of the enhancer downstream of MYC occur, on average, 112 bp downstream of TSS's on chromosome 8. (D) Examples of raw CATCH-seq histograms (background subtracted) demonstrating the locations of peaks overlapping with enhancer marks. Additionally, the peak at the E2F5 promoter demonstrates the specificity of CATCH, as no other gene promoters in the region are positive for long-distance interactions. (E) IPA analysis detailing specific gene promoters that demonstrated physical interaction with the enhancer downstream of MYC. The most significantly enriched process and disease enriched within this dataset was homologous recombination and gastric carcinoma, respectively.

FIG. 12. SIAH2 (vehicle-treated) enhancer CATCH-seq interaction peaks significantly overlap with enhancer marks and ER-binding sites compared to statistically random control; the frequency of these overlaps increase with peak height. The same pattern is observed with estradiol treatment in FIG. 4E.

FIG. 13. Tables containing the list of genes that had both significant CATCH-seq signal peaks within 2 kb of their promoters, and were also identified by SEEK as being transcriptionally co-expressed. NB: SIAH2 is independently identified (w/estradiol treatment).

FIG. 14. (A) Graph detailing the number of SEEK genes ‘predicted’ by the vehicle76 treated or estradiol-treated versions of SIAH2 enhancer CATCH-seq genome-wide. (B) IPA showing the topmost statistically significant and enriched pathway for each small gene subset identified by CATCH-seq/SEEK overlap: female genital tract cancer (estradiol treated) and endometrial cancer (vehicle treated).

FIG. 15. Analysis of CTCF binding adjacency/overlap with CATCH peaks. Numbers 1 and 2 denote replicates for GRB7 and EIF4A1. (A, left) Control GRB7 promoter region CATCH-seq interaction peaks do not significantly overlap with CTCF-binding sites and have very modest overlap with ER-binding sites compared to statistically random control; the frequency of these overlaps does not increase with peak height. (A, right) EIF4A1 promoter region CATCH-seq interaction peaks do not significantly overlap with CTCF-binding sites, but have significant overlap with ER-binding sites compared to statistically random control; the frequency of the latter overlaps increases as a function of peak height. (B) SIAH2 downstream enhancer region CATCH-seq interaction peaks do not significantly overlap with CTCF-binding sites, but have significant overlap with ER-binding sites compared to statistically random control; the frequency of the latter overlaps increases as a function of peak height. (C) MYC downstream enhancer region CATCH-seq interaction peaks show modest overlap with CTCF-binding sites, but have significant overlap with ER-binding sites compared to statistically random control; the frequency of both overlaps increases, to some degree, as a function of peak height.

FIG. 16. Tables 1-4.

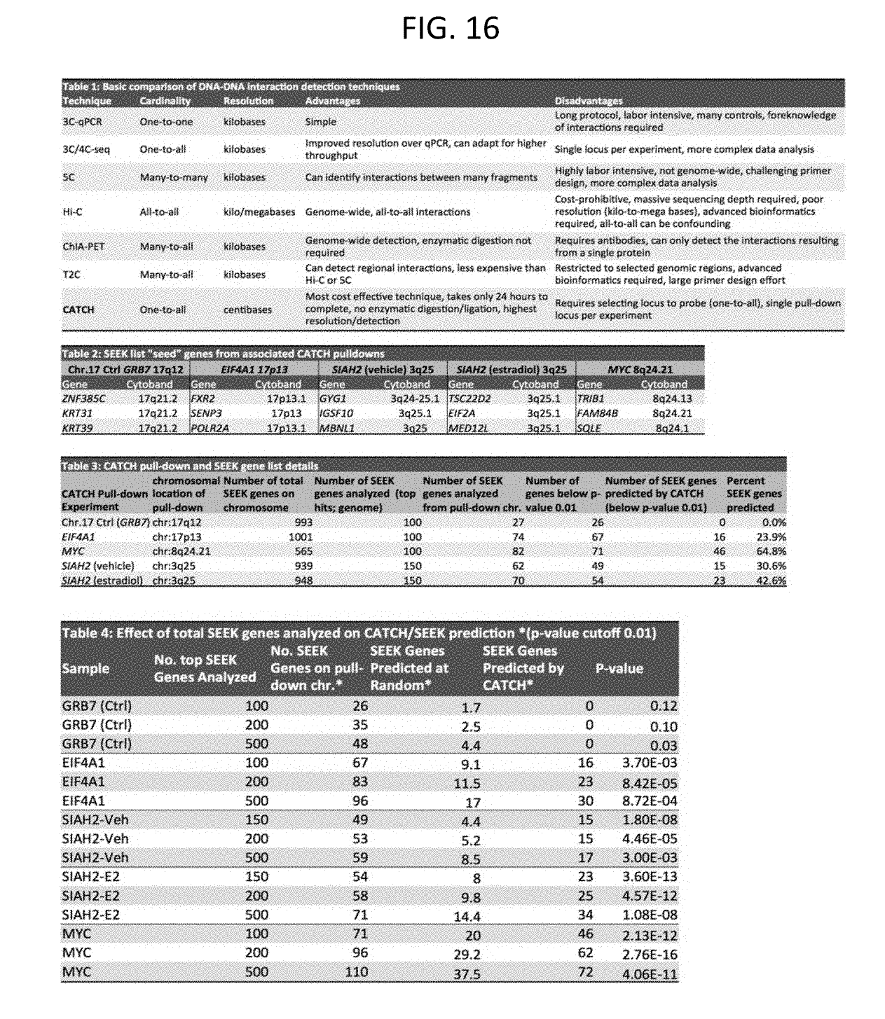

### DEFINITIONS

The terminology used herein is for the purpose of describing the particular embodiments only, and is not intended to limit the scope of the embodiments described herein. Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. Thus, for example, reference to “an oligonucleotide” is a reference to one or more oligonucleotides and equivalents thereof known to those skilled in the art, and so forth.

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

### DETAILED DESCRIPTION

Provided herein are compositions and methods for identifying endogenous DNA-DNA interactions. In particular, compositions and methods are provided for performing Capture of Associated Targets on CHromatin (CATCH) assays which use efficient capture and enrichment of specific genomic loci of interest through hybridization and subsequent purification via complementary oligonucleotides, without the need for enzymatic digestion or ligation steps.

CATCH is highly reproducible, does not require enzymatic digestion or ligation, exhibits base pair resolution, and is completed in less than 24 hours. In addition to capture and analysis of a single locus, CATCH finds use in assessing genome-wide relationships when paired with next-generation sequencing. CATCH finds use in analysis of a single chromosome, or, with deeper sequencing coverage, genome-wide CATCH is achieved.

CATCH utilizes a labeled capture oligonucleotide. In some embodiments, the label is a tag or handle that allows for capture of the oligonucleotide and a target hybridized thereto. In some embodiments, a capture oligonucleotide is a biotinylated capture oligonucleotide. In experiments conducted during development of embodiments herein, biotin was attached at the 5′ end of the oligonucleotide using a 15-atom triethylene glycol (TEG) spacer that eliminated steric hindrance between the biotin moiety and the target DNA-protein complexes, allowing full accessibility to the streptavidin magnetic beads. Other linkers and linker lengths find use in embodiments herein. In some embodiments, a desthiobiotin moiety is used, allowing for a gentler elution from the beads with the addition of excess biotin.

Experiments conducted during development of embodiments herein demonstrated that CATCH is capable of detecting previously unreported long-distance chromatin interactions. Earlier studies demonstrated an interaction between the intronic ERE^{B} and the ERE downstream of the SIAH2 gene (ref. 16; incorporated by reference in its entirety) and CATCH confirms this interaction. However, CATCH-seq also demonstrated the existence of a highly complex web of interactions between the downstream enhancer of SIAH2 and multiple enhancers and promoters spanning the entirety of chromosome 3; this finding also held true for loci on chromosomes 8 and 17. While the data presented herein support the concept that gene promoters are being physically linked, the biochemical data paint a slightly different picture. Traditionally, gene promoters are thought to span approximately 5 kbp upstream of a gene's TSS, however CATCH-seq data demonstrates that the average chromatin looping interaction involving the TSS region of genes occurs between 50-200 bp downstream of the TSS. The identification of such interactions has not been demonstrated by existing techniques.

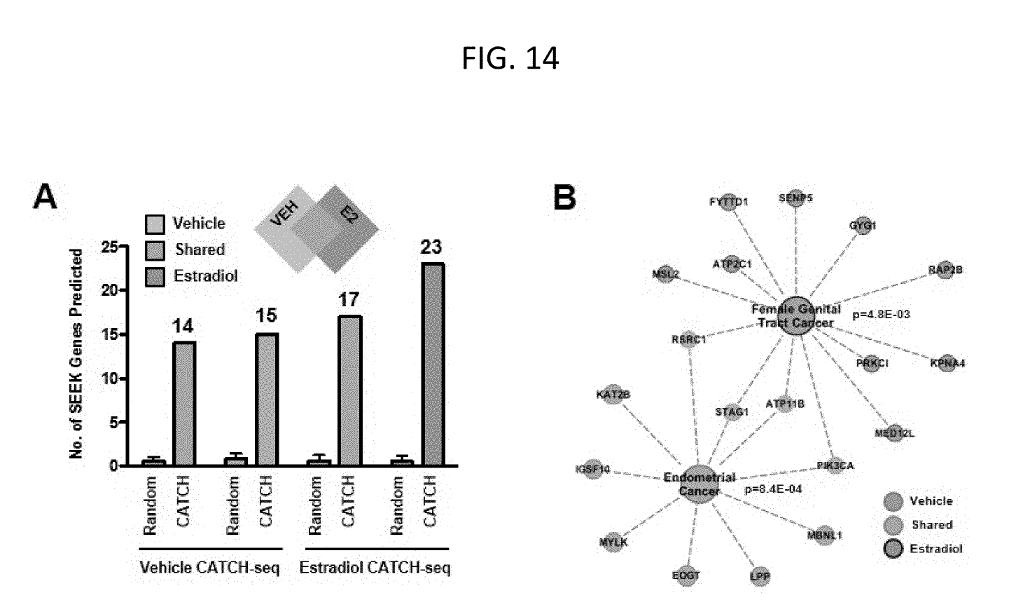

Experiments conducted using CATCH indicate that single enhancers regulate a host of genes, even at linear distances of multiple millions of base pairs. The data demonstrate that subsets of genes, spanning entire chromosomes, physically associate with the same enhancer, and that a highly significant portion of those genes are co-expressed within the cell. In relation to these transcriptionally-associated DNA-DNA interactions, the CTCF protein has been implicated in mediating such looping (ref 28; incorporated by reference in its entirety). Experiments were conducted during development of embodiments herein to assess to what degree CATCH peaks and CTCF binding sites were adjacent or overlapping in T47D cells. There was very little overlap found between CTCF binding sites and CATCH peaks (FIG. 14).

In some embodiments, methods of fixing protein-protein, protein-DNA, protein-RNA, RNA-DNA, RNA-RNA, DNA-DNA interactions are employed. In some embodiments, fixing reagents are added to cells to fix (e.g., crosslink such interactions. In certain embodiments, a sample is fixed with formalin, formaldehyde, ethanol, methanol, picric acid, etc.

In some embodiments, methods herein comprise a step of shearing (fragmenting) nucleic acids (e.g., fixed nucleic acids). Nucleic acids may be sheared (fragmented) by physical (mechanical) or chemical means, for example, by sonicating, shearing, or enzymatically digestion or chemical cleavage of DNA.

In some embodiments, tagged oligonucleotides are hybridized to target sequences. In some embodiments, the oligonucleotides are complementary (e.g., 100%, >95%, >90%, >85%, >80%, >75%, >70%, >65%, >60%, >55%) to target sequences. In some embodiments, the tag on the oligonucleotide facilitates capture of the hybridized complex by a complimentary capture moiety. Suitable tags include biotin, glutathione, a hexahistidine tag, a FLAG tag and digoxigenin, which can captured by streptavidin, glutathione S-transferase, an anti-his antibody, an anti-FLAG antibody and anti-digoxigenin, respectively. In some embodiments, the capture moiety is attached to a solid surface, bead (eg., magnetic bead), etc.

Some embodiments herein comprise methods for analyzing target sequences and any associated nucleic acid sequences. Such processes may include nucleic acid amplification, hybridization, mass analysis, sequencing, etc.

In some embodiments, methods of detection/analysis comprise nucleic acid amplification, for example, by polymerase chain reaction (PCR). The PCR process is well known in the art (U.S. Pat. Nos. 4,683,195, 4,683,202, and 4,800,159). To briefly summarize PCR, nucleic acid primers, complementary to opposite strands of a nucleic acid amplification target nucleic acid sequence, are permitted to anneal to the denatured sample. A DNA polymerase (typically heat stable) extends the DNA duplex from the hybridized primer. The process is repeated to amplify the nucleic acid target. If the nucleic acid primers do not hybridize to the sample, then there is no corresponding amplified PCR product. In this case, the PCR primer acts as a hybridization probe.

In PCR, the nucleic acid probe can be labeled with a tag as discussed before. Most preferably the detection of the duplex is done using at least one primer directed to the target nucleic acid. In yet another embodiment of PCR, the detection of the hybridized duplex comprises electrophoretic gel separation followed by dye-based visualization.

DNA amplification procedures by PCR are well known and are described in U.S. Pat. No. 4,683,202. Briefly, the primers anneal to the target nucleic acid at sites distinct from one another and in an opposite orientation. A primer annealed to the target sequence is extended by the enzymatic action of a heat stable DNA polymerase. The extension product is then denatured from the target sequence by heating, and the process is repeated. Successive cycling of this procedure on both DNA strands provides exponential amplification of the region flanked by the primers.

Amplification is then performed using a PCR-type technique, that is to say the PCR technique or any other related technique. Two primers, complementary to the target nucleic acid sequence are then added to the nucleic acid content along with a polymerase, and the polymerase amplifies the DNA region between the primers.

The expression “specifically hybridizing in stringent conditions” refers to a hybridizing step in the process of the invention where the oligonucleotide sequences selected as probes or primers are of adequate length and sufficiently unambiguous so as to minimize the amount of non-specific binding that may occur during the amplification. The oligonucleotide probes or primers herein described may be prepared by any suitable methods such as chemical synthesis methods.

Hybridization is typically accomplished by annealing the oligonucleotide probe or primer to the DNA under conditions of stringency that prevent non-specific binding but permit binding of this DNA which has a significant level of homology with the probe or primer.

Among the conditions of stringency is the melting temperature (Tm) for the amplification step using the set of primers, which is in the range of about 55° C. to about 70° C.

Typical hybridization and washing stringency conditions depend in part on the size (i.e., number of nucleotides in length) of the DNA or the oligonucleotide probe, the base composition and monovalent and divalent cation concentrations (Ausubel et al., 1997, eds Current Protocols in Molecular Biology).

In some embodiments, methods herein involve sequencing target and/or captured nucleic acid sequences. Nucleic acid molecules may be sequence analyzed by any number of techniques. The analysis may identify the sequence of all or a part of a nucleic acid. Illustrative non-limiting examples of nucleic acid sequencing techniques include, but are not limited to, chain terminator (Sanger) sequencing and dye terminator sequencing, as well as “next generation” sequencing techniques. In some embodiments, RNA is reverse transcribed to cDNA before sequencing. A number of DNA sequencing techniques are known in the art, including fluorescence-based sequencing methodologies (See, e.g., Birren et al., Genome Analysis: Analyzing DNA, 1, Cold Spring Harbor, N.Y.; herein incorporated by reference in its entirety). In some embodiments, automated sequencing techniques understood in that art are utilized. In some embodiments, the systems, devices, and methods employ parallel sequencing of partitioned amplicons (PCT Publication No: WO2006084132 to Kevin McKernan et al., herein incorporated by reference in its entirety). In some embodiments, DNA sequencing is achieved by parallel oligonucleotide extension (See, e.g., U.S. Pat. No. 5,750,341 to Macevicz et al., and U.S. Pat. No. 6,306,597 to Macevicz et al., both of which are herein incorporated by reference in their entireties). Additional examples of sequencing techniques include the Church polony technology (Mitra et al., 2003, Analytical Biochemistry 320, 55-65; Shendure et al., 2005 Science 309, 1728-1732; U.S. Pat. No. 6,432,360, U.S. Pat. No. 6,485,944, U.S. Pat. No. 6,511,803; herein incorporated by reference in their entireties) the 454 picotiter pyrosequencing technology (Margulies et al., 2005 Nature 437, 376-380; US 20050130173; herein incorporated by reference in their entireties), the Solexa single base addition technology (Bennett et al., 2005, Pharmacogenomics, 6, 373-382; U.S. Pat. No. 6,787,308; U.S. Pat. No. 6,833,246; herein incorporated by reference in their entireties), the Lynx massively parallel signature sequencing technology (Brenner et al. (2000). Nat. Biotechnol. 18:630-634; U.S. Pat. No. 5,695,934; U.S. Pat. No. 5,714,330; herein incorporated by reference in their entireties), the Adessi PCR colony technology (Adessi et al. (2000). Nucleic Acid Res. 28, E87; WO 00018957; herein incorporated by reference in its entirety), and suitable combinations or alternative thereof.

A set of methods referred to as “next-generation sequencing” techniques have emerged as alternatives to Sanger and dye-terminator sequencing methods (Voelkerding et al., Clinical Chem., 55: 641-658, 2009; MacLean et al., Nature Rev. Microbiol., 7: 287-296; each herein incorporated by reference in their entirety). Next-generation sequencing (NGS) methods share the common feature of massively parallel, high-throughput strategies, with the goal of lower costs and higher speeds in comparison to older sequencing methods. NGS methods can be broadly divided into those that require template amplification and those that do not. Sequencing techniques that finds use in embodiments herein include, for example, Helicos True Single Molecule Sequencing (tSMS) (Harris T. D. et al. (2008) Science 320:106-109; U.S. Pat. No. 7,169,560), Lapidus et al. (U.S. patent application number 2009/0191565), Quake et al. (U.S. Pat. No. 6,818,395), Harris (U.S. Pat. No. 7,282,337), Quake et al. (U.S. patent application number 2002/0164629), and Braslaysky, et al., PNAS (USA), 100: 3960-3964 (2003), each of which is incorporated by reference in their entireties); 454 sequencing (Roche) (Margulies, M et al. 2005, Nature, 437, 376-380; incorporated by reference in its entirety); SOLiD technology (Applied Biosystems); Ion Torrent sequencing (U.S. patent application numbers 2009/0026082, 2009/0127589, 2010/0035252, 2010/0137143, 2010/0188073, 2010/0197507, 2010/0282617, 2010/0300559), 2010/0300895, 2010/0301398, and 2010/0304982; incorporated by reference in their entireties); Illumina sequencing; real-time (SMRT) technology of Pacific Biosciences; nanopore sequencing (Soni G V and Meller A. (2007) Clin Chem 53: 1996-2001; incorporated by reference in its entirety); use of a chemical-sensitive field effect transistor (chemFET) array to sequence DNA (for example, as described in US Patent Application Publication No. 20090026082; incorporated by reference in its entirety); other sequencing techniques (e.g., NGS techniques) understood in the field, or alternatives or combinations of the above techniques find use in embodiments herein.

### EXPERIMENTAL

### Methods

### Exemplary CATCH Protocol

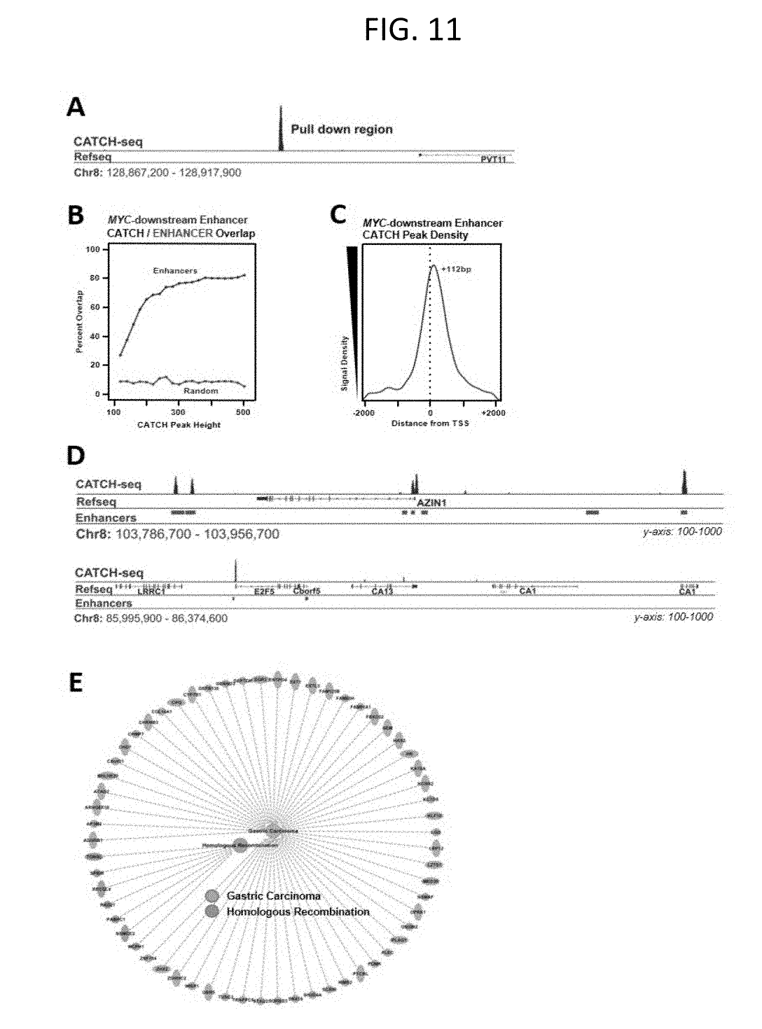

MCF-7 or T-47D cells (log-phase growth, approximately 1×10⁶ cells per sample) were fixed for 6 minutes at room temperature with a final concentration of 1% formaldehyde (fresh single-use vials) at approximately 50% culture confluency. Crosslinking was quenched for 10 minutes at room temperature by the addition of Tris-HCl pH 8.0 to a final concentration of approximately 0.125 M. Next the cells were harvested via scraping in 1 ml of PBS into a 1.5 ml eppendorf tube, and then spun at 250×g for 8 minutes (centrifugation steps were carried out in a standard tabletop microfuge). The supernatant was aspirated and the cells were resuspended in 500 μl of cold Nuclear Isolation Buffer supplemented with a protease inhibitor cocktail (Calbiochem). The samples were dounce homogenized 20 times with a tight-fitting pestle and centrifuged again for 10 minutes at 750×g to pellet nuclei. The supernatant was aspirated and the nuclear pellets were resuspended in 100 μl of CATCH Buffer supplemented with protease inhibitor cocktail. The samples were sonicated for 2 cycles (HIGH, 30s on/off) of 8 minutes each in a Diagenode BioRuptor® sonication device, and the cellular debris was pelleted by centrifugation at 24,000×g for 15 minutes at 4° C. Sonication efficiency was then assessed on a 1.5% (w/v) agarose gel. Genomic DNA fragments should largely fall between 100 and 500 bp. Next, the sheared chromatin sample was incubated at 58° C. for 5 minutes to unmask biotin on endogenous proteins. Then, 10 μl of pre-equilibrated (in CATCH Buffer) streptavidin magnetic beads (Thermo Scientific) were added to each sample. The samples were incubated for 1 hour at room temperature while gently rotating. Next, the magnetic beads were extracted and the supernatant was transferred to a clean PCR tube. To each sample, specific biotinylated oligonucleotide probe (Integrated DNA Technologies) was added to a final concentration of approximately 300 nM. The probe was then hybridized by incubating the samples as follows: 25° C. for 2 minutes, 81° C. for 4 minutes (denaturation), 72° C.-42° C. decreasing gradient (12 seconds per degree), 42° C. for 30 minutes (hybridization), followed by storage of the sample at 25° C. During testing, denaturation temperatures below 75° C. or above 85° C. were detrimental to oligonucleotide annealing or long-range interaction detection, respectfully; impact on interaction detection at 81° C. was undetectable. The hybridized sample was then transferred to a new 1.5 ml eppendorf tube and any unhybridized biotinylated oligo was removed with an Illustra Sephacryl (S-400HR) spin column, according to manufacturer's instruction. The cleared product was again transferred to a new 1.5 ml eppendorf containing 300 μl of nuclease-free H₂O. Next, 25 μl of pre-equilibrated (in CATCH Buffer) streptavidin magnetic beads were added to each sample. The samples were incubated at room temperature for 1 hour while gently rotating. The beads from each sample were then immobilized on a magnetic stand and washed 5 times in CATCH Buffer at 42° C. while shaking at 1000 RPM in a thermomixer. The beads were then resuspended in 150 μl of De-crosslinking Buffer supplemented with 5 μl of 20 mg/ml Proteinase K. The sample was incubated at 55° C. for 30 minutes to while shaking at 1000 RPM on a thermomixer, followed by incubation at 65° C. overnight on the same thermomixer. Finally, the sample was incubated at 100° C. for 60 seconds to destroy any remaining biotin-streptavidin binding and elute the DNA from the magnetic beads. The supernatant was immediately transferred to a new 1.5 ml eppendorf tube. The DNA was then purified using phenol-chloroform-isoamyl alcohol, and then precipitated in 100% ethanol using glycogen (Thermo Scientific) as a carrier. The DNA was pelleted by spinning at 24,000×g for 25 minutes at room temperature, and resuspended in TE Buffer. An overview comparison of CATCH and other chromosome capture methods is available in Table 1 (See FIG. 11).

### Exemplary CATCH Biotinylated Oligo Design

Biotinylated oligonucleotides were ordered from Integrated DNA Technologies, using the TEG-Biotin modification on the 5′ end of the oligo. All oligos were designed with Primer3 version 4.0 to be between 23 and 25 nucleotides in length, with a Tₘ as close to 63° C. as possible. Testing multiple oligonucleotides, it was found that those biotinylated oligos targeted to regions approximately 150 bp from the targeted protein-binding site gave the most reliable data. The oligos were resuspended at 1 μg/μl in TE buffer and stored at −20° C. until use.

### Cell Culture

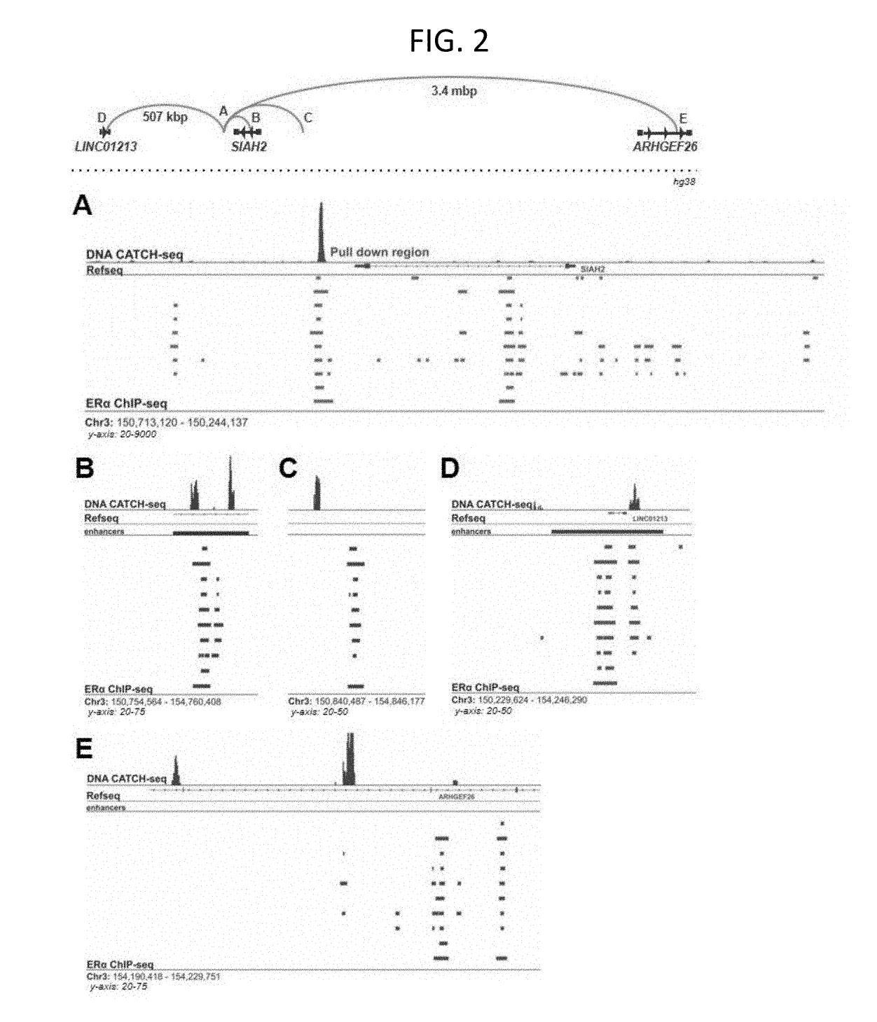

MCF-7 (ATCC; HTB-22) and T-47D (ATCC; HTB-133) cells were maintained in phenol red-free RPMI 1640 with L-glutamine supplemented with 10% (v/v) heat-inactivated fetal bovine serum and 100 U/ml penicillin-streptomycin. Cells were housed at 37° C. in 5% CO₂ for a maximum of 12 passages after being purchased directly from the American Type Culture Collection (ATCC). The cells used in FIG. 2 for the validation of CATCH-seq (when compared to ChIA-PET) were MCF-7 at passage 3 after being purchased directly from the ATCC (HTB-22). According to the ATCC (MCF-7): cytogenetic analysis yielded a modal chromosome number of 82, with a range of 66 to 87. The stemline chromosome numbers ranged from hypertriploidy to hypotetraploidy, with the 2S component occurring at 1%. There were 29 to 34 marker chromosomes per S metaphase; 24 to 28 markers occurred in at least 30% of cells, and generally one large submetacentric (M1) and 3 large subtelocentric (M2, M3, and M4) markers were recognizable in over 80% of metaphases. No DM were detected. Chromosome 20 was nullisomic and X was disomic.

### CLOVER Analysis

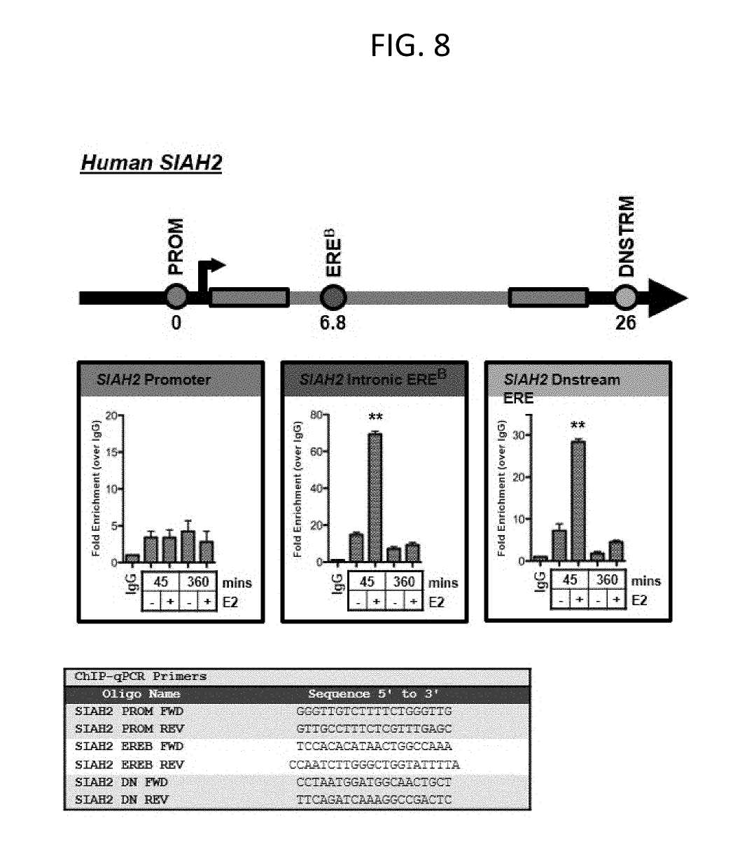

Using the freely available CLOVER (zlab.bu.edu/clover) program (ref. 30; incorporated by reference in its entirety) according to the specified instructions, full-site estrogen response elements were identified within and around the SIAH2 gene ±100kb. The resulting potential binding sites were then cross-referenced to previously identified ER-binding sites within MCF-7 cells (ref 31; incorporated by reference in its entirety). The NCBI36/hg18 build of the human genome was used. Full-site EREs identified by CLOVER and/or positively correlated with previous data were then used in the subsequent ChIP and CATCH assays. These sites are detailed in FIG. 8.

### Chromatin Immunoprecipitation (ChIP)

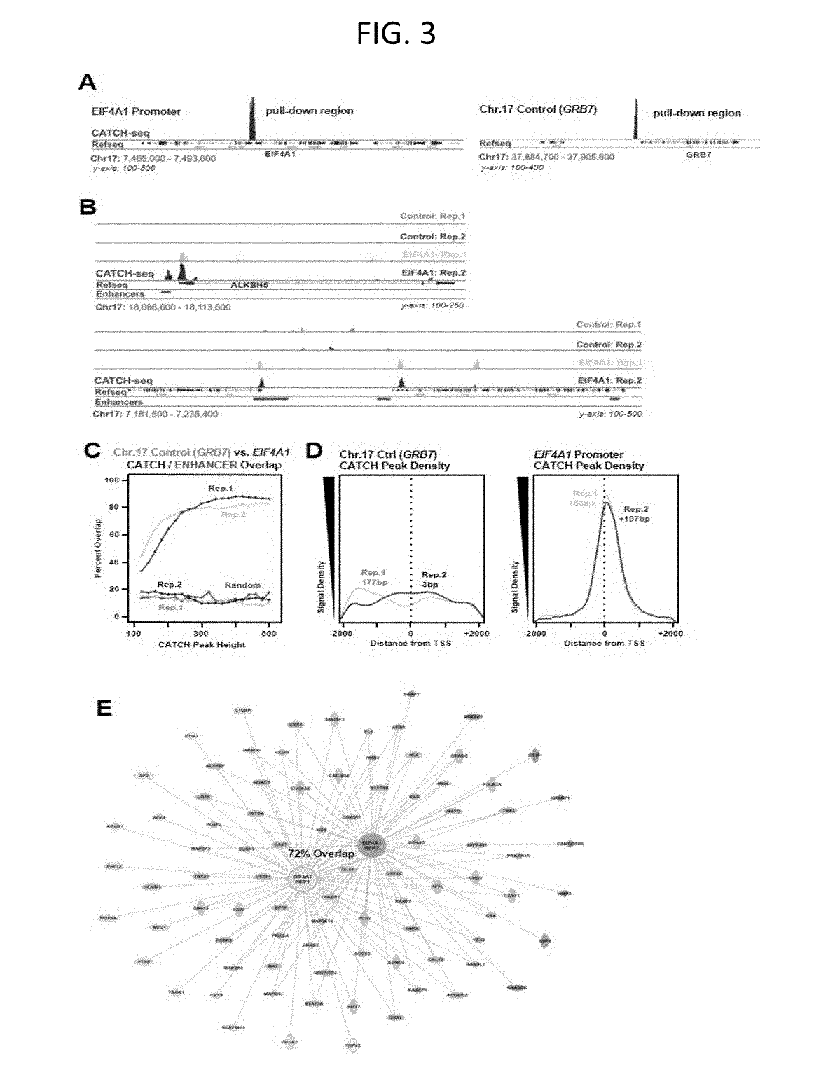

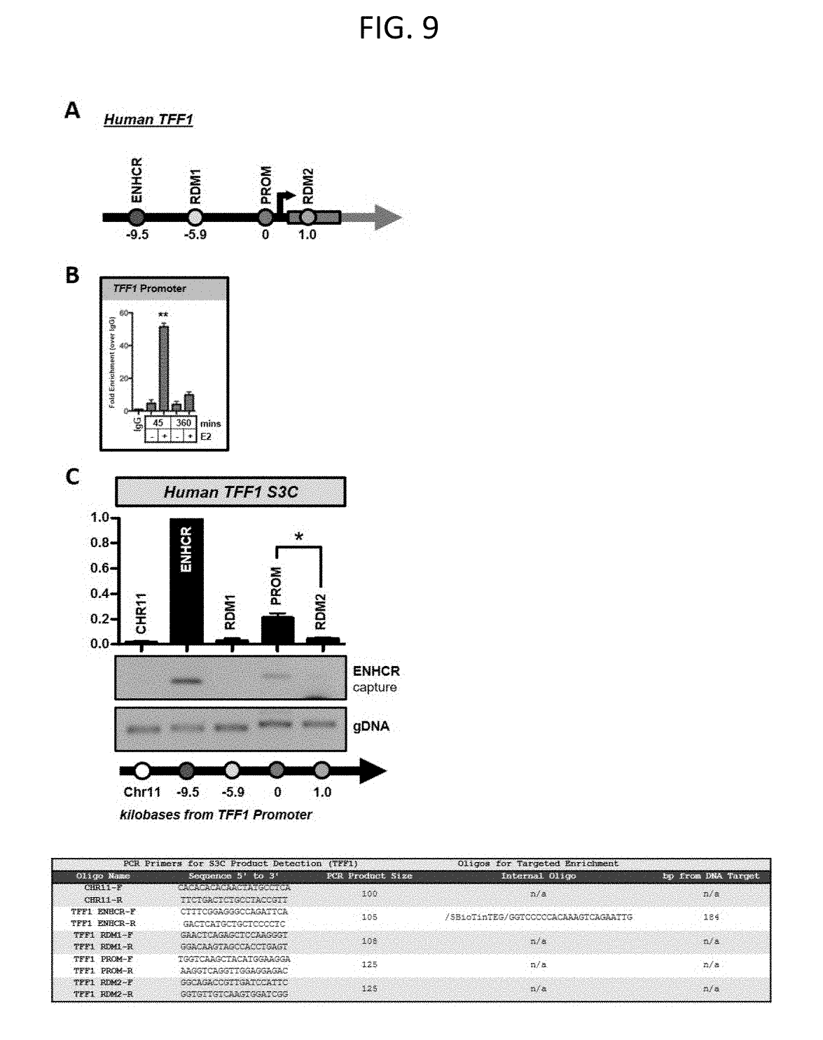

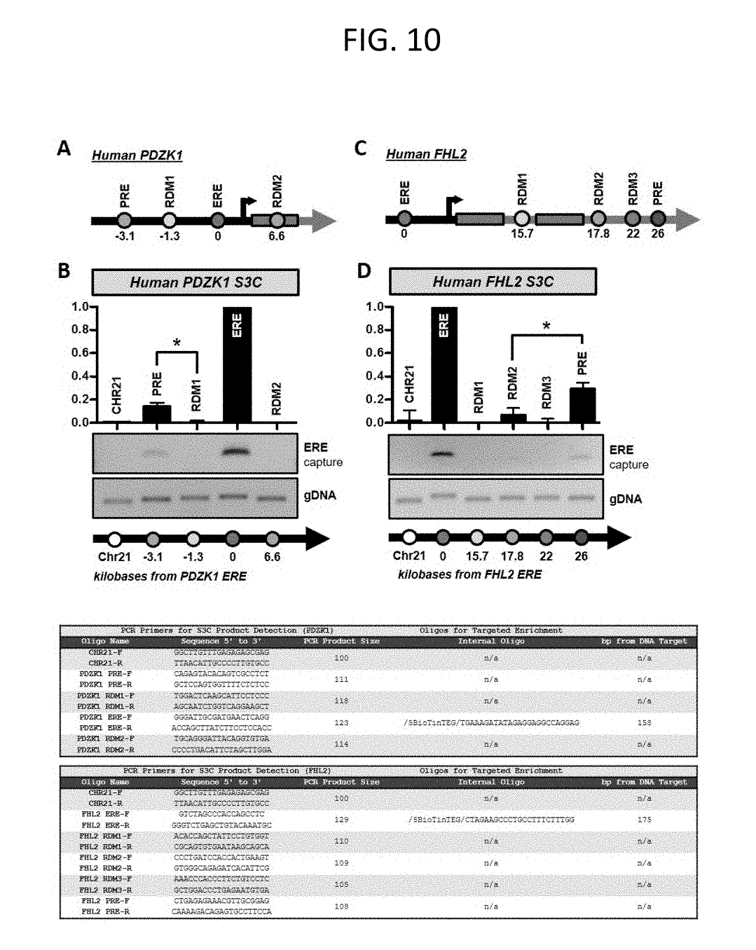

Cells were fixed with 1% formaldehyde for 10 minutes at room temperature. Reaction was quenched with glycine, cells were centrifuged to pellet, and resuspended in ChIP Lysis Buffer (10 mM Tris pH 8.0, 10 mM NaCl, 5 mM EDTA, 1% NP-40, 1% SDS, 0.5% Deoxycholate) supplemented with protease inhibitors. The cell slurry was incubated for 10 minutes on ice and then sonicated for 3 cycles (HIGH, 30s on/off) of 7 minutes each in a Diagenode BioRuptor® sonication device, and the cellular debris was pelleted by centrifugation at 24,000×g for 15 minutes at 4° C. Sonication efficiency was then assessed on a 1.5% (w/v) agarose gel. Genomic DNA fragments largely fell between 100 and 700 bp. Next, the sheared chromatin sample was diluted to 1 ml in ChIP Dilution Buffer (17 mM Tris pH 8.0, 33 mM NaCl, 1% SDS, 0.5% NP-40) supplemented with protease inhibitors. Here, 10% of total volume was taken as input. Then, 2 μg of anti-ERα (Santa Cruz Biotech, HC-20) or rabbit IgG antibody was added to each sample, and the samples were rotated overnight at 4° C. Next, magnetic protein-G Dynabeads (Invitrogen) were washed once in PBS supplemented with 5% BSA and resuspended in ChIP dilution buffer. Then 30 μl of the pre-washed beads were added to each sample, and the samples were rotated at 4° C. for 2 hours. The beads were then washed consecutively in ChIP Wash Buffer I (20 mM Tris pH 8.0, 150 mM NaCl, 2 mM EDTA, 1% NP-40, 1% SDS), ChIP Wash Buffer II (20 mM Tris pH 8.0, 500 mM NaCl, 2 mM EDTA, 1% NP-40, 1% SDS), ChIP Wash Buffer III (20 mM Tris pH 8.0, 250 mM LiCl, 1 mM EDTA, 1% NP-40, 1% Deoxycholate), and TE buffer. The beads were then resuspended in 100 μl freshly made ChIP Elution Buffer (200 μl of 10% SDS and 0.168 g of NaHCO₃ in 2 ml of H₂O). Next the samples were incubated at 65° C. for 15 minutes to elute the complex from the beads. That process was repeated and the eluates were combined. Finally, 8 μl of 5.0 M NaCl was added to each sample (including input samples) and they were incubated overnight at 65° C. Samples were incubated with RNase and Proteinase K prior to processing with QIAquick® PCR purification kit (Qiagen) according to manufacturers instructions. PCR Primers for individual ChIP experiments are detailed in FIGS. 9 and 10. ChIP for ERα (Santa Cruz Biotechnology, sc-542) and H3K4me1 (Millipore, 07-436)/H3K27ac (Millipore, 07-360) (FIGS. 3 & 4) were performed as above and followed by Illumina next-generation sequencing at the UChicago Sequencing Facility. For each ChIP, 2 μg of antibody was used per experiment.

### PCR Quantification (ImageJ)

PCR products were diluted in 6× Orange G loading buffer and run at 100V for 28 minutes on a 1.5% agarose gel with ethidium bromide (ladder was Bioline EasyLadder I). The resulting gel was imaged under ultraviolet light, and the individual bands were quantified via ImageJ using the measure function. First, the background value for each band was taken. Next, the value of the band itself was taken, and the background value was subtracted from the value of the band. Each resulting value was then normalized to the value of the targeted pull down in the experiment, such that the value of the pull down became 1.0. This ensured subtraction of background variation and random variation in pull down efficiency. The resulting values were then plotted as mean with error bars of SEM.

### Creation of Sequencing Libraries

10 μl of DNA from CATCH final elution was immediately (without freezing) put through second strand synthesis protocol using NEBNext Module #E6111S according to manufacturer instructions. Nucleic Acid binding beads were AMPure XP #A63881, purchased from Agencourt. Next, the DNA template was made into a sequencing library using the KAPA Biosystems library kit #KK8232 following manufacturer instruction. The KAPA kit was critical as it produces a library with fewer “bead swap” steps, allowing you to retain a better DNA template yield and thus make a library from less starting material. Sequencing depth for each library varied between ˜15 to ˜24 million reads: GRB7 replicates 1 and 2 had 16.0 and 22.9 million reads, respectively, MYC had 16.1 million reads, EIF4A1 replicates 1 and 2 had 18.6 and 24.2 million reads, respectively, SIAH2 vehicle-treated had 16.2 million reads, and SIAH2 estradiol-treated had 15.1 million reads.

### Creation of SEEK Lists

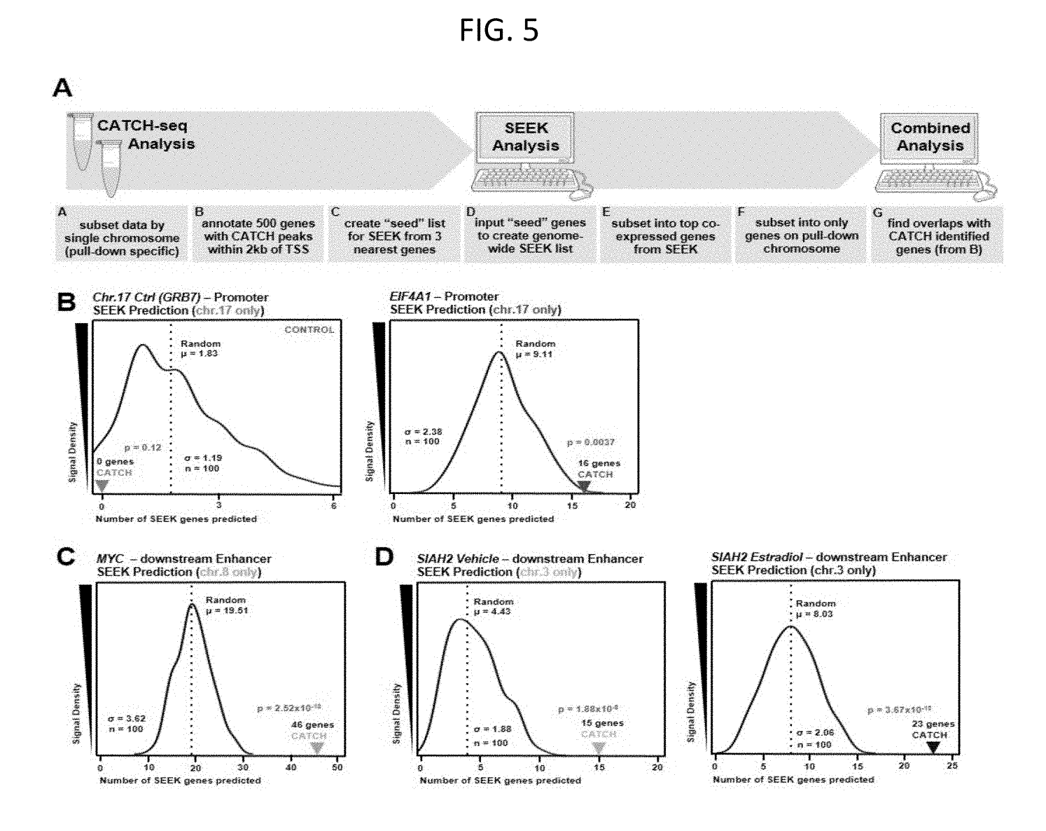

The SEEK algorithm (seek.princeton.edu) is a web application stemming from research done at Princeton University (ref. 17; incorporated by reference in its entirety). SEEK allows a number of genes as input to determine a ranked-order list by co-expression. This co-expression rank is a comprehensive analysis based on over 5000 independent microarray and RNA sequencing data sets. In T-47D cells: to create each SEEK list, 3 “seed” input genes were selected with the following rules: (a) the gene must have a CATCH-seq peak within 2 kb of its TSS and the peak must be above background, (b) the peak near the TSS of the gene must be one of the top 500 (in height) such peaks on the chromosome, and (c) the gene must not be the primary target of the enhancer, according to our study (e.g. SIAH2 was not used as a “seed” gene, despite being identified in the associated CATCH experiment wherein the downstream enhancer of SIAH2 was captured). Using those guidelines, the three gene promoters nearest the CATCH capture site were chosen as input to determine the list of co-expressed genes via SEEK (FIG. 11, Table 2). The total number of genes in the SEEK data base is 17,857, and each final SEEK list used only the top 100/150 (based on co-expression value). In analyses where a random SEEK list was required (FIG. 5B-D): to determine the random distribution, the co-expression rankings of the SEEK gene list was permuted (at random) and its overlap was calculated with the CATCH-seq gene list. The lengths of the gene lists used to calculate the random was kept the same as the ones used to calculate the prediction by CATCH. Subsequently, mean and standard deviation of the random distribution was calculated and p value was determined using a t distribution. Only genes from the specific pull-down chromosome were used in this calculation. This was also considered optimal, as we were testing the viability of CATCH to predict gene co-expression, not the inverse. A flow-chart detailing the steps of this analysis can be found in FIG. 5A, and a more detailed table of CATCH prediction of SEEK genes is available in FIG. 11, Table 3.

### Data Analysis

Data analysis was performed using R version 3.2.2 within RStudio. ERα ChIP-seq BED file data from MCF-7 cells (FIG. 2A-E) was obtained from ENCODE at the UCSC genome browser. Those data sets, from top-to-bottom were provided by: Barton, Brown, Carroll, Chinnaiyan, Hurtado (Carroll), Liu, Odom, Stunnenberg, Weisz, and White labs.

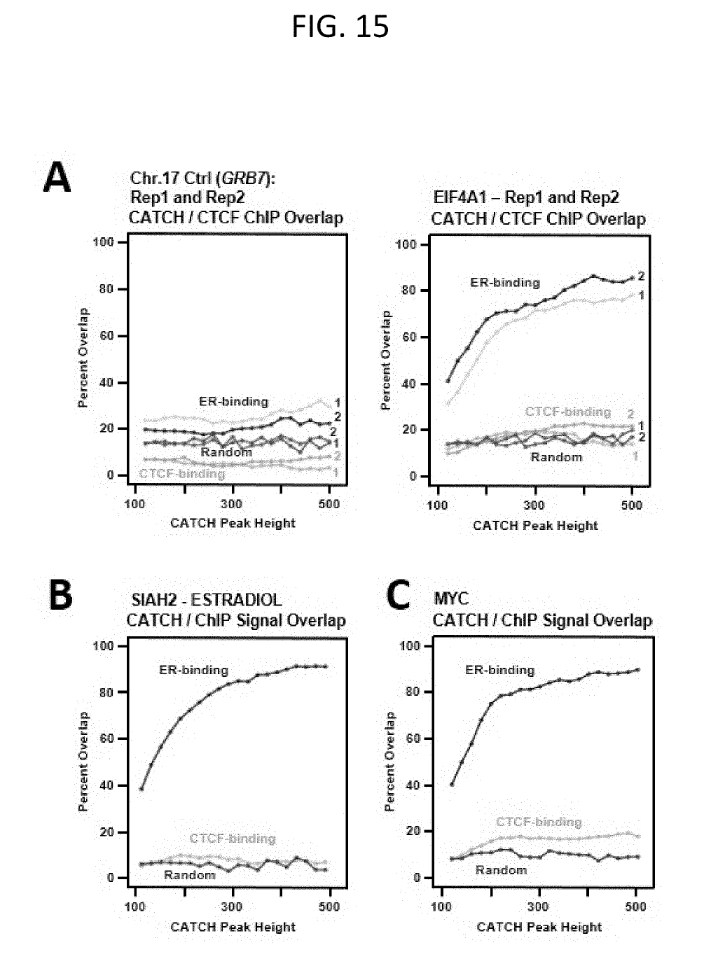

CATCH-seq Data Sets: Each CATCH-seq experiment was done alongside an unfixed control experiment using an identical capture oligo. The raw data underwent FASTQ Groomer processing, before being aligned to the hg19 build of the chromsome of interest using Bowtie 2. Firstly, an unfixed control pulldown (CATCH experiment, minus any fixation method) using the same biotinylated oligonucleotide is normalized from the experimental pulldown data by aligning unfixed control reads (.bam) and ‘subtracting’ from the experimental reads, directly, to remove any background signal using the bamCompare function in Galaxy deepTools2. The data was then subsetted by individual chromosome (e.g. chr3 for SIAH2, chr8 for MYC, etc.), in R, and then by signal threshold (the signal threshold is determined based on the number of CATCH interactions desired to discover). The identification of that threshold determined the signal strength at which CATCH peaks were defined as peaks; with the threshold determined, those CATCH peaks that satisfied the signal strength threshold in the BIGWIG of respective pull-down were next called. Next, gene promoters that had peaks within 2 k of their promoters were then annotated and considered as interactions with the pulldown locus. The top 500 (highest peaks) genes were then assessed to determine the three closest CATCH-identified genes to the pull-down. These three genes were used as the “seed” for creating the SEEK list, which is described above. In the case of FIG. 15, a continuous variable of peak numbers was discovered to determine the p value at which CATCH's ability to predict SEEK gene co-expression became non-significant.

Enhancers: To determine the location of Enhancers, ChIP-seq data from both H3K4me1 and H3K27ac was used. Peaks were called using the MACS (version 1.4.1; p-value cutoff 1e-05; MFOLD range 32, 128; fixed background lambda) function of Galaxy, and peak locations of at least 1 bp overlap between H3K4me1 and H3K27ac signal were identified. The combined distance of the two peaks was merged into a single peak, denoted an Enhancer, and made into a BED file for further use.

ER-binding: locations of ER binding were determined as above, instead using ERα ChIP-seq data and without combining any other data sets.

CTCF-binding: locations of CTCF binding were from CTCF-binding data from T47D cells (GEO accession: GSM803348).

CATCH-seq gene identification: to ensure that only the most significant CATCH-seq peaks were used for analysis, gene promoters with peaks within 2 kb of their TSS were identified. If multiple peaks occurred near a TSS, only the most robust peak was considered. Then, the 500 genes with the strongest CATCH-seq signal peaks were identified for use in subsequent analyses. CATCH-Enhancer adjacency/overlap: CATCH-seq signal strength (peak height) ranging from 100 to 500 was analyzed. CATCH peaks were determined by filtering based on the strength of the signal in BIGWIG files of the respective pull-down. Any CATCH-seq peak within 2 kb of an Enhancer region (as defined above) was considered to be adjacent or overlapping, thus achieving our criteria for being considered an overlap in these analyses. TSS Density Plot: the plot(density(x)) function in R was used to plot CATCH-seq signal strength at locations within 2 kb up- and down-stream of every TSS on a chromosome (chr3 for SIAH2, chr17 for EIF4A1, and chr8 for MYC). That signal density plot was used to determine the average location of signal “peaks” near gene TSS.

### Results

### Capture of Associated Targets on Chromatin (CATCH) Identifies Long-Distance Genomic Interaction

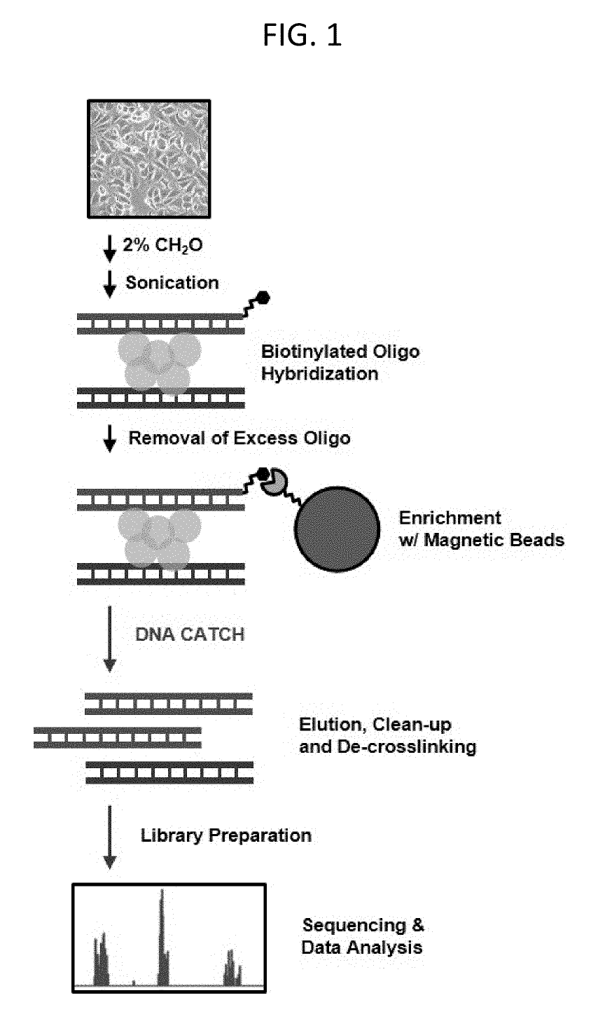

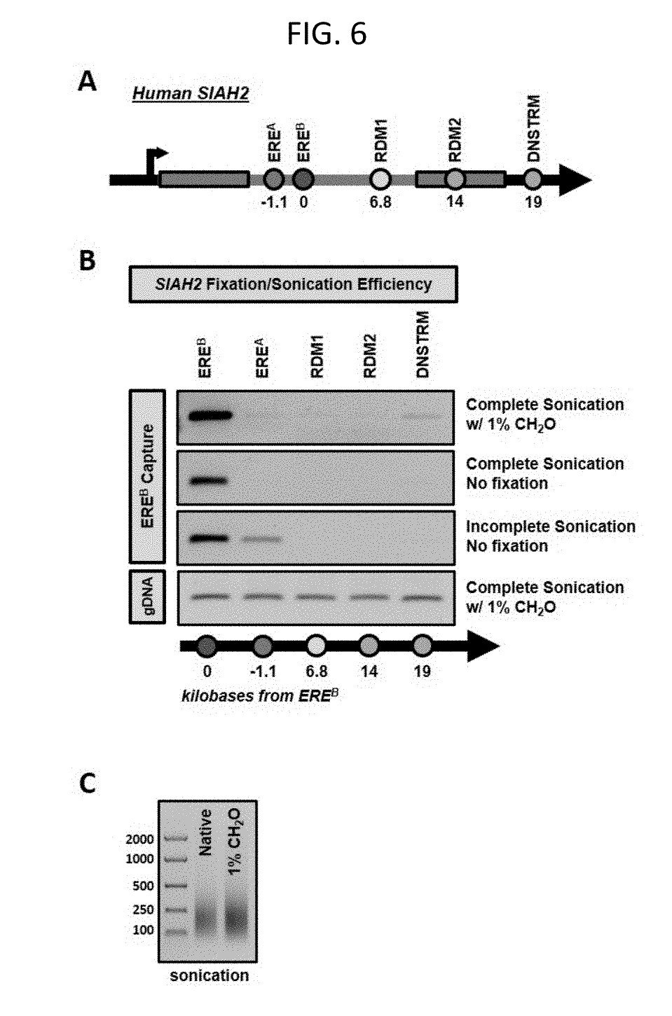

A flowchart representing the CATCH methodology and process is shown in FIG. 1. The use of sonication is a relatively unbiased method of DNA shearing. To demonstrate CATCH sonication and fixation efficiency, an intronic region of the SIAH2 gene that had previously been shown to interact with a region downstream of the gene (Fullwood et al., 2009; incorporated by reference in its entirety) was targeted; this intronic region is an established ER-binding site and was designated ERE^{B}. The human SIAH2 gene has two exons and a single intron located on the minus strand of chromosome 3; ERE^{B} is located within the intron, approximately 6.8 kilobases from the SIAH2 promoter. Random genomic loci were also tested for interactions within SIAH2. The tested target or random loci ranged in distance between 1.1 and 19 kilobases from ERE^{B} (FIG. 6a). Probing for the presence of each locus in the pool of gDNA that was subjected to the hybridization and capture steps resulted in the amplification of each locus, as expected; this result demonstrated that formaldehyde fixation and sonication does not destroy or bias the availability of genomic loci (FIG. 6b). Importantly, without formaldehyde fixation, it was possible to pull down additional loci with ERE^{B} only if the sample was incompletely sonicated (FIG. 6b), due to the proximity of the two loci on the linear genome. This result demonstrated that any interacting loci seen with the addition of formaldehyde could be interpreted as transient physical interactions that require static fixation to capture, consistent with the current model of genomic looping mediated via protein complexes. Efficient sonication of DNA was not impacted by formaldehyde treatment (FIG. 6c).

### CATCH-Seq Recapitulates ERα ChIA-PET Data

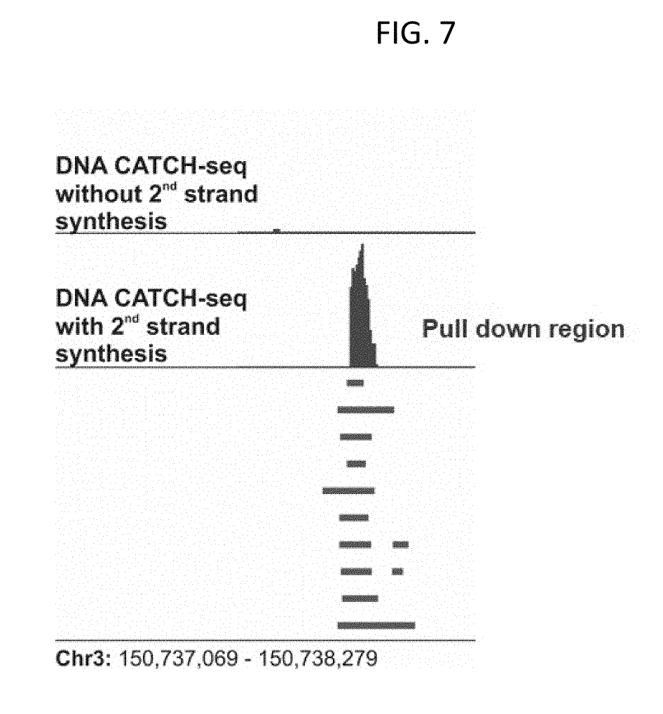

To demonstate CATCH compatibility with next generation sequencing, the established enhancer region downstream of the SIAH2 gene was targeted. CATCH followed by next generation sequencing (CATCH-seq) demonstrated that the oligo-targeted downstream ERE (pull down region) was highly enriched when compared to any other genomic site (FIG. 2a). Due to the nature of the assay, the capture of this region left the DNA of the targeted pull-down locus single stranded, and second-strand synthesis was used prior to sequencing to retain its integrity (FIG. 7).

SIAH2 is an estradiol-responsive ER target gene with multiple putative EREs located within and adjacent to the gene. In a study focused on identifying functional ER binding sites, the authors predicted that the intronic region of SIAH2 contributed to the transcriptional regulation of the gene (ref 18; incorporated by reference in its entirety). However, the promoter region of SIAH2 does not contain a recognizable ERα binding site, and chromatin immunoprecipitation (ChIP) experiments confirmed that ERα binding was nearly undetectable at the promoter, nor was it responsive to E2 (FIG. 8). In contrast, both of the tested EREs showed significant increases in ERα occupancy after 45 minutes of E2 treatment (FIG. 8). These data indicate that the intronic and downstream EREs were interacting with the SIAH2 promoter to influence transcription.

In concordance with these data, ERα ChIA-PET analysis of the SIAH2 gene demonstrated interaction between a portion of the intron and an enhancer region directly downstream of the gene (ref. 16; incorporated by reference in its entirety). Additionally, the interaction between the downstream enhancer of SIAH2 and multiple other long-distance genomic loci (visualized through Washington University in St. Louis's WashU Epigenome browser; epigenomegateway.wustl.edu; FIG. 2, top).

In order to demonstrate that CATCH-seq recapitulates data obtained with previously validated techniques, experiments were conducted to identify ERα-mediated genomic looping interactions attributed to the downstream enhancer of SIAH2. Each of the four long-distance interactions tested were positively demonstrated by CATCH-seq. Interaction with the intronic ERE of SIAH2 (distance: 17 kb) was demonstrated here in MCF-7 cells (FIG. 2a). Long distance looping with the ERE upstream of SIAH2 (distance: 103 kb) was also positively demonstrated, despite its interaction signal appearing approximately 2 kb from the previously-identified ERα binding site (FIG. 2b). The downstream ERE of SIAH2 has also been shown to interact with an ERα binding site (distance: 507 kb) within an enhancer region adjacent to a long non-coding RNA known as LINC01213. This interaction was also recapitulated using CATCH-seq (FIG. 2c), however the interaction signal was lower than that of the other interactions tested. The presence of an interaction within an intron of the ARHGEF26 gene (distance: 3.4 mb) was also shown to occur as previously identified (FIG. 2d).

Another canonical ERE/Promoter interaction on the TFF1 gene was confirmed (FIG. 9), and CATCH was used to demonstrate interaction between progestin response elements (PREs) and EREs at both the PDZK1 and FHL2 genes (FIG. 10). These findings demonstrate that CATCH-seq is capable of reliably identifying previously-validated chromatin interaction data.

### EIF4A1 Promoter CATCH-Seq Demonstrates Specificity and Reproducibility

In order to demonstrate the specificity of CATCH at the level of sequencing, multiple biological replicates were sequenced separately and compared to a ‘random’ locus capture on the same chromosome (chromosome 17). It has been suggested that the promoter region of the human EIF4A1 gene is involved in multiple chromatin interactions with neighboring loci (ref 19; incorporated by reference in its entirety). In contrast, while a number of interactions occur adjacent to the GRB7 promoter, it was used as a control pull-down because none of the interactions identified near GRB7 looked to directly involve the promoter. While the direct capture of both regions of interest was successful (FIG. 3a), the CATCH-seq peaks (representing regions physically interacting with the pull-down region) identified with specific capture of the EIF4A1 promoter gave highly robust output signal comparative to GRB7, hereafter referred to as control (FIG. 3b). Many CATCH signal peaks were noted to overlap with histone marks of active enhancers; enhancers were defined as ChIP-seq positive areas of overlapping H3K27ac and H3Kme1 in T47D cells (FIG. 3b). Analysis of these data for the entirety of chromosome 17 revealed that both EIF4A1 CATCH-seq replicates had approximately 40% signal overlap with enhancers at lower peak thresholds, but as the CATCH-seq peak threshold was increased (e.g., stronger CATCH-seq signal) this overlap increased to over 80% (FIG. 3c). By contrast, both control capture replicates did not significantly overlap with enhancers above random. These results indicated that enhancers play a significant role in determining sites of chromatin looping and contribute to gene expression, thus involving gene promoters. CATCH-seq signals within 2000 bp of a transcription start site (TSS) on chromosome 17 were plotted by density. While the control CATCH-seq produced only random noise, the EIF4A1 promoter CATCH-seq revealed a striking peak near the TSS of promoters, suggesting significant DNA-DNA interaction enrichment (FIG. 3d). Additionally, when the replicates were evaluated via Ingenuity Pathway Analysis, the two EIF4A1 CATCH-seq replicates had 72% overlap in identified signal peaks, and a similarly high degree of overlap in identified gene promoters, indicating an exceptionally high degree of reproducibility among biological replicates for sequencing experiments (FIG. 3e).

To determine if these observations held true on a different chromosome, an enhancer downstream of the MYC gene was interrogated in similar fashion (FIG. 11a). As previously, a large proportion of CATCH-seq signal resulting from the MYC-downstream enhancer capture overlapped with enhancer marks, and this proportion trended exponentially upward with signal strength, growing to over 80% at higher peak thresholds (FIG. 11b). CATCH-seq peaks within 2000 bp of a TSS were also centered near the transcription start sites on chromosome 8, again falling approximately 112 bp downstream of the TSS, reinforcing the data from FIG. 3D (FIG. 11c). These data again indicated that enhancers play a critical role in DNA-DNA interactions that occur near gene promoters, as can be seen in this interaction between the captured enhancer and the AZIN1 promoter (FIG. 11d, top). While the CATCH-seq data demonstrated that the captured enhancer downstream of MYC was interacting with more than a single promoter, not every gene promoter harbors an interaction signal, suggesting that CATCH-seq is capturing authentic DNA-DNA interaction. One example is the E2F5 promoter, which demonstrates strong interaction with the enhancer downstream of MYC at the exclusion of other genes in the area (FIG. 11d, bottom). Ingenuity Pathway Analysis found the processes of gastric carcinoma and homologous recombination to be the top pathways regulated by this enhancer (FIG. 11e). These data indicate that the captured enhancer downstream of MYC is capable of physically interacting with a host of additional enhancers and gene promoters across chromosome 8, and that the genes involved in these interactions can be linked to common disease/biochemical processes.

### ER Activation Alters the Chromatin Interactions of Downstream Enhancer of SIAH2

Estradiol is a powerful genome-wide transcriptional inducer via estrogen receptor activation. SIAH2 transcription is upregulated upon ER activation.

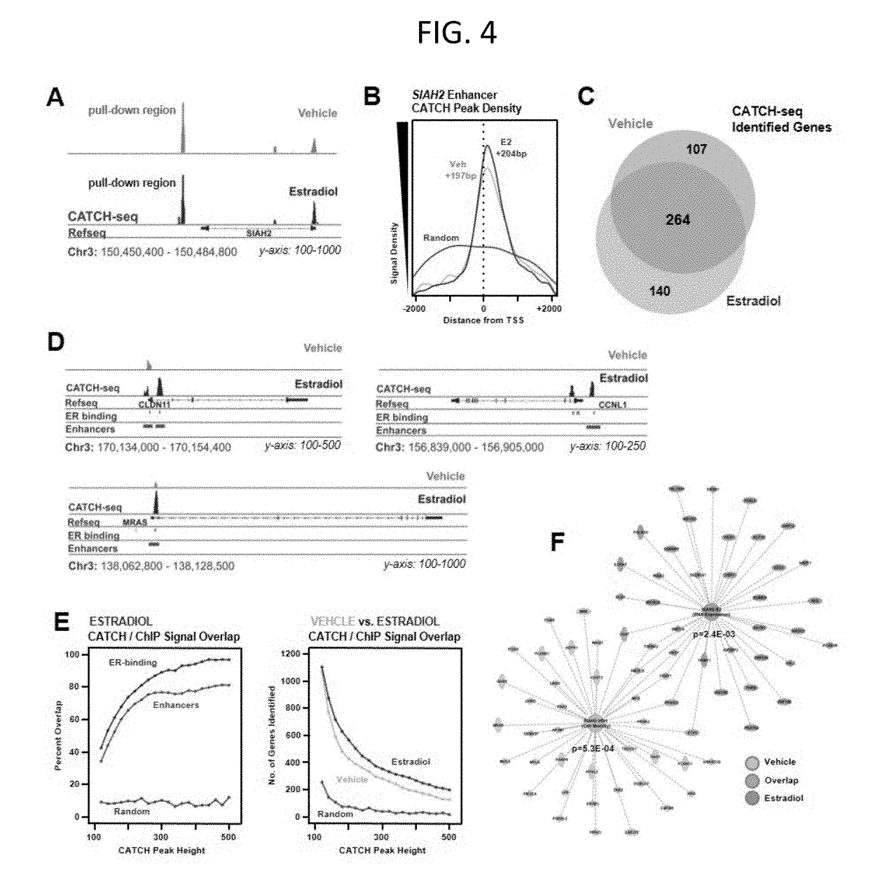

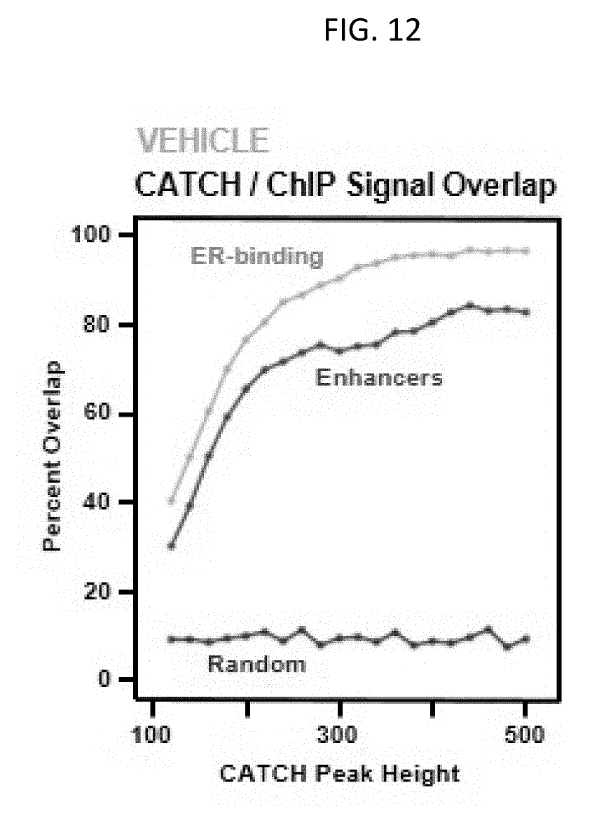

Despite SIAH2 transcription being activated by estradiol treatment, both vehicle and estradiol treatments showed DNA-DNA interaction between the downstream enhancer (pull-down region) and the SIAH2 intron and promoter (FIG. 4a). As with the other CATCH-seq pull-downs on chromosomes 8 and 17, the CATCH-seq signal density within 2000 bp of a TSS peaked significantly (compared to randomized distribution of the data set) near the transcription start sites of chromosome 3, averaging about 200 bp downstream for both vehicle and estradiol treatments (FIG. 4b). When comparing genes on chromosome 3 whose promoters were identified as interacting with the downstream enhancer of SIAH2, vehicle and estradiol treatments shared 52% overlap (264 genes), indicating that estradiol treatment was not responsible for completely rearranging genomic structure (FIG. 4c). However, estradiol treatment lost 107 enhancer-promoter interactions from vehicle baseline, while it gained 140, demonstrating a critical plasticity in genomic architecture that could potentially play a role in altering transcriptional programs and activity. Many of these types of interactions are illustrated in FIG. 4D, showing individual examples of enhancer-promoter interactions being gained upon estradiol treatment, as well as significant overlaps with CATCH-seq signals and enhancer sites on chromosome 3 (FIG. 4d). A large proportion of the CATCH-seq signal resulting from the SIAH2 downstream enhancer capture overlapped with enhancer marks. A similar trend was observed for overlap with locations of ER-binding in both the presence (FIG. 4e, left) and absence (FIG. 12) of estradiol, and at higher CATCH-seq signal thresholds this overlap reached well over ninety percent. While sites of ER-binding were highly correlated to sites of chromatin looping for this particular enhancer in both the presence and absence of estradiol, it was clear that estradiol had a significant impact on chromatin architecture. Not only did estradiol trigger a chromosome-wide alteration in the genes interacting with the downstream enhancer of SIAH2, but the total number of genes identified by CATCH-seq was higher (at all thresholds) in the presence of estradiol (FIG. 4e, right). These results indicate a great deal of plasticity in DNA-DNA interactions in response to estradiol. This was further illustrated via Ingenuity Pathway Analysis, which elucidated highly distinct subsets of genes in the presence and absence of estradiol (FIG. 4f). The RNA expression process was significantly enriched upon estradiol treatment (FIG. 4f, blue), whereas there was no enrichment of this pathway in the absence of estradiol (FIG. 4f, gray); these findings support the function of estradiol as a genome-wide gene expression modulator. These findings indicate the potential for malleable transcriptional foci, containing the interactions of many genes at once; many enhancer-enhancer or enhancer-promoter interactions within such a hub would stable, but some could form or dissipate based on changing biochemical stimuli.

CATCH-Seq Predicts Correlation with Gene Expression Using the SEEK Algorithm

In order to test whether a single enhancer is capable of interacting with, and altering the transcription of, multiple gene targets on the same chromosome, the SEEK algorithm (search-based exploration of expression compendia; seek.princeton.edu) was employed. SEEK determines gene expression correlation by weighting available gene expression datasets based on input genes of interest (ref 17 incorporated by reference in its entirety); using this weighted correlation aggregation method, it calculates relative gene co-expression amongst those datasets. If CATCH-seq is identifying DNA-DNA interactions at gene promoters that led to the alteration of transcriptional expression of that gene, CATCH-seq data should be significantly more proficient at predicting co-expression of gene cohorts than random. If the list of gene promoters identified via CATCH-seq is significantly enriched (over random) for genes that are also transcriptionally co-expressed, it would indicate that the long-distance genomic interactions of a single enhancer are capable of influencing gene expression patterns, not just the transcriptional output of a single gene. The top 500 gene promoters for each CATCH-seq experiment were identified. Next, SEEK lists were created using a “seed list”; a three gene subset specific to each CATCH-seq experiment; each SEEK list was then sorted based on the highest co-expression value. The top 100 co-expressed genes from the pull-down chromosome of interest for each CATCH-seq experiment were denoted the SEEK list. A flow chart describing the processing of each data set can be found in FIG. 5a. The EIF4A1 promoter CATCH-seq identified 16 genes also present on the SEEK list; by contrast, a random SEEK list created from only genes found on that chromosome (chromosome 17) only identified an average of 9 genes in common with the CATCH-seq experiment (FIG. 5b, right). The control capture on chromosome 17 was incapable of identifying any genes on the SEEK list (FIG. 5b, left). Strikingly, capture of the enhancer downstream of MYC yielded an overlap of 46-out-of-100 genes from its unique SEEK co-expression list (FIG. 5c). Similarly, both conditions of SIAH2 downstream enhancer CATCH predicted significantly more genes than would be expected at random (FIG. 5d), as the CATCH prediction results were statistically outside the entire histogram of random prediction. These results demonstrated that CATCH-seq was capable of identifying DNA-DNA interactions involving subsets of gene promoters that are transcriptionally linked (co-expressed). In order to show that this predictive power was not a function of using too selective SEEK gene list, additional SEEK genes (100, 200, 500) were used in the CATCH/SEEK analysis. In all cases, CATCH was still able to predict significantly more SEEK genes than random (FIG. 11, Table 4).

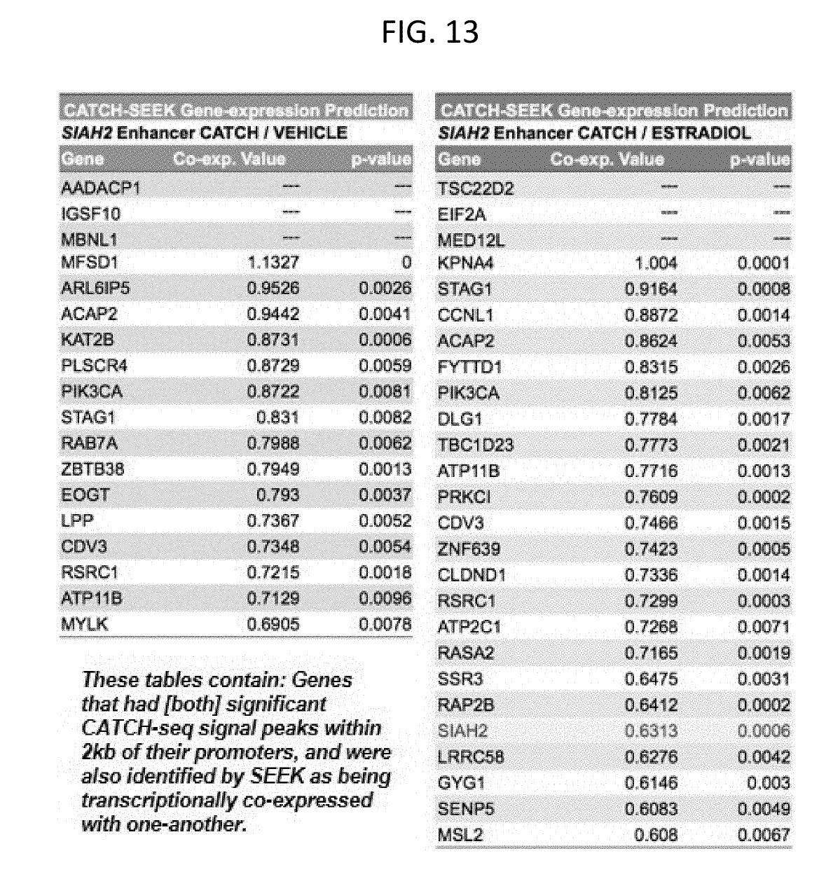

The presence of estradiol induced changes in the DNA-DNA interactions of the downstream enhancer of SIAH2 (FIG. 4). In order to determine whether CATCH-seq interactions were able to predict gene co-expression under these conditions, unique 150 gene SEEK lists were created for the vehicle- and estradiol-treated samples of the downstream enhancer of SIAH2 CATCH-seq experiment. The majority of genes predicted to be co-expressed were identified in both the vehicle- (93%) and estradiol-treated (74%) conditions (FIG. 5c). However, consistent with the data that demonstrated the presence of estradiol increased the total number of chromatin interactions (FIG. 4E), estradiol treatment also resulted in more CATCH-seq predictive power. In total, the estradiol-treated condition of the downstream enhancer of SIAH2 CATCH-seq was able to predict the co-expression of 23 genes (FIG. 5c), compared to an average of 0.64 genes predicted at random, while the vehicle-treated condition of the same experiment predicted the co-expression of only 15 genes (FIG. 5c). SIAH2 was identified as one of the top co-expressed genes upon estradiol treatment, but not vehicle (FIG. 11, Table 5). Because the captured region in this particular CATCH-seq experiment is a known enhancer of SIAH2, these results indicate that the CATCH-seq interactions were making biologically meaningful predictions of transcriptional co-expression. Ingenuity Pathway Analysis was also done on these highly limited subsets of 15 and 23 genes for vehicle- and estradiol-treatment, respectively (FIG. 13a). Both subsets of genes were significantly linked to female-specific cancers (FIG. 13, indicating that the downstream enhancer of SIAH2 is both hormone responsive, and capable of influencing the expression of a host of genes involved in these processes. Despite the majority of ‘predicted’ genes being common to both vehicle and estradiol treatment, the most significant enriched pathways were defined largely by unique genes (FIG. 13b). Together, these data indicate that single enhancers are capable of regulating a host of transcriptionally co-expressed genes, even at linear distances of multiple millions of base pairs.

### REFERENCES

The following references, some of which are cited above by number, are herein incorporated by reference in their entireties.
1. Li, G. & Reinberg, D. Chromatin higher-order structures and gene regulation. Curr. Opin. Genet. Dev. 21, 175-186 (2011).
2. Clapier, C. R. & Cairns, B. R. The Biology of Chromatin Remodeling Complexes. Annu. Rev. Biochem. 78, 273-304 (2009).
3. Rands, C. M., Meader, S., Ponting, C. P. & Lunter, G. 8.2% of the Human Genome Is Constrained: Variation in Rates of Turnover across Functional Element Classes in the Human Lineage. PLoS Genet 10, e1004525 (2014).
4. Consortium, T. E. P. An integrated encyclopedia of DNA elements in the human genome. Nature 489, 57-74 (2012).
5. Pennacchio, L. A. et al. In vivo enhancer analysis of human conserved non-coding sequences. Nature 444, 499-502 (2006).
6. Lam, M. T. Y., Li, W., Rosenfeld, M. G. & Glass, C. K. Enhancer RNAs and regulated transcriptional programs. Trends Biochem. Sci. 39, 170-182 (2014).
7. Thurman, R. E. et al. The accessible chromatin landscape of the human genome. Nature 489, 75-82 (2012).
8. Shlyueva, D., Stampfel, G. & Stark, A. Transcriptional enhancers: from properties to genome-wide predictions. Nat. Rev. Genet. 15, 272-286 (2014).
9. Kadauke, S. & Blobel, G. A. Chromatin loops in gene regulation. Biochim. Biophys. Acta 1789, 17-25 (2009).
10. Marsden, M. P. & Laemmli, U. K. Metaphase chromosome structure: evidence for a radial loop model. Cell 17, 849-858 (1979).
11. Dekker, J., Rippe, K., Dekker, M. & Kleckner, N. Capturing Chromosome Conformation. Science 295, 1306-1311 (2002).
12. Gavrilov, A. A., Golov, A. K. & Razin, S. V. Actual Ligation Frequencies in the Chromosome Conformation Capture Procedure. PLoS ONE 8, e60403 (2013).
13. Wit, E. de & Laat, W. de. A decade of 3C technologies: insights into nuclear organization. Genes Dev. 26, 11-24 (2012).
14. Behling, K. C. et al. Increased SIAH expression predicts ductal carcinoma in situ (DCIS) progression to invasive carcinoma. Breast Cancer Res. Treat. 129, 717-724 (2011).
15. Ross-Innes, C. S. et al. Differential oestrogen receptor binding is associated with clinical outcome in breast cancer. Nature 481, 389-393 (2012).
16. Fullwood, M. J. et al. An Oestrogen Receptor α-bound Human Chromatin Interactome. Nature 462, 58-64 (2009).
17. Zhu, Q. et al. Targeted exploration and analysis of large cross-platform human transcriptomic compendia. Nat. Methods 12, 211-214 (2015).
18. Vega, V. B. et al. Multiplatform genome-wide identification and modeling of functional human estrogen receptor binding sites. Genome Biol. 7, R82 (2006).
19. Li, G. et al. Extensive Promoter-Centered Chromatin Interactions Provide a Topological Basis for Transcription Regulation. Cell 148, 84-98 (2012).
20. Kulaeva, O. I., Nizovtseva, E. V., Polikanov, Y. S., Ulianov, S. V. & Studitsky, V. M. Distant Activation of Transcription: Mechanisms of Enhancer Action. Mol. Cell. Biol. 32, 4892-4897 (2012).
21. Kalhor, R., Tjong, H., Jayathilaka, N., Alber, F. & Chen, L. Genome architectures revealed by tethered chromosome conformation capture and population-based modeling. Nat. Biotechnol. 30, 90-98 (2012).
22. Dixon, J. R. et al. Chromatin architecture reorganization during stem cell differentiation. Nature 518, 331-336 (2015).
23. Dostie, J. et al. Chromosome Conformation Capture Carbon Copy (5C): a massively parallel solution for mapping interactions between genomic elements. Genome Res. 16, 1299-1309 (2006).
24. van Berkum, N. L. et al. Hi-C: a method to study the three-dimensional architecture of genomes. J. Vis. Exp. JoVE (2010). doi:10.3791/1869
25. Kolovos, P. et al. Targeted Chromatin Capture (T2C): a novel high resolution high throughput method to detect genomic interactions and regulatory elements. Epigenetics Chromatin 7,10 (2014).
26. Kidder, B. L., Hu, G. & Zhao, K. ChIP-Seq: technical considerations for obtaining high-quality data. Nat. Immunol. 12, 918-922 (2011).
27. Déjardin, J. & Kingston, R. E. Purification of Proteins Associated with Specific Genomic Loci. Cell 136, 175-186 (2009).
28. Ong, C.-T. & Corces, V. G. CTCF: An Architectural Protein Bridging Genome Topology and Function. Nat. Rev. Genet. 15, 234-246 (2014).
29. CTCF Binding Polarity Determines Chromatin Looping: Molecular Cell. Available at: http://www.cell.com/molecular-cell/abstract/S1097-2765(15)00762-5. (Accessed: 16 Mar. 2016)
30. Frith, M. C. et al. Detection of functional DNA motifs via statistical over-representation. Nucleic Acids Res. 32, 1372-1381 (2004).
31. Hurtado, A., Holmes, K. A., Ross-Innes, C. S., Schmidt, D. & Carroll, J. S. FOXA1 is a key determinant of estrogen receptor function and endocrine response. Nat. Genet. 43, 27-33 (2011).

1. A method comprising:
(a) fixing a cell population to capture nucleic acid-protein-nucleic acid interactions;
(b) sonicating the cell population to shear the nucleic acid into small fragments;
(c) hybridizing nucleic acid target sequences to a labeled oligo;
(d) separating the hybridized nucleic acid from unhybridized nucleic acid, thereby enriching for target sequences and associated protein-nucleic acid complexes;
(e) de-crosslinking; and
(f) analyzing target sequences and any associated nucleic acid sequences.

2. The method of claim 1, wherein the cell population is formaldehyde fixed.

3. The method of claim 1, wherein the labeled oligo is a biotinylated oligo.

4. The method of claim 3, wherein the hybridized nucleic acid is separated from the unhybridized nucleic acid using streptavidin-linked magnetic beads.

5. The method of claim 1, wherein analyzing target sequences and any associated nucleic acid sequences comprises performing PCR amplification.

6. The method of claim 1, wherein analyzing target sequences and any associated nucleic acid sequences comprises next-generation sequencing.

7. The method of claim 6, wherein the method results in the identification of DNA sequences physically associated with the target acid target sequences (via proteins).

8. The method of claim 1, wherein the method does not comprise an enzymatic digestion step.

9. The method of claim 1, wherein the method does not comprise a ligation step.

10. The method of claim 1, wherein the target nucleic acid and/or associated nucleic acid is DNA.

11. The method of claim 1, wherein the target nucleic acid and/or associated nucleic acid is RNA.

12. A composition, system, or kit for performing the method of claim 1.


SEQUENCE LISTING (39 sequences)

SEQ ID NO: 1 - DNA, 20 bp, Homo sapiens
gggttgtcttttctgggttg

SEQ ID NO: 2 - DNA, 20 bp, Homo sapiens
gttgcctttctcgtttgagc

SEQ ID NO: 3 - DNA, 20 bp, Homo sapiens
tccacacataactggccaaa

SEQ ID NO: 4 - DNA, 22 bp, Homo sapiens
ccaatcttgggctggtatttta

SEQ ID NO: 5 - DNA, 20 bp, Homo sapiens
cctaatggatggcaactgct

SEQ ID NO: 6 - DNA, 20 bp, Homo sapiens
ttcagatcaaaggccgactc

SEQ ID NO: 7 - DNA, 21 bp, Homo sapiens
cacacacacaactatgcctca

SEQ ID NO: 8 - DNA, 19 bp, Homo sapiens
ttctgactctgcctacgtt

SEQ ID NO: 9 - DNA, 20 bp, Homo sapiens
ctttcggagggccagattca

SEQ ID NO: 10 - DNA, 19 bp, Homo sapiens
gactcatgctgctcccctc

SEQ ID NO: 11 - DNA, 20 bp, Homo sapiens
gaactcagagctccaagggt

SEQ ID NO: 12 - DNA, 20 bp, Homo sapiens
ggacaagtagccacctgagt

SEQ ID NO: 13 - DNA, 21 bp, Homo sapiens
tggtcaagctacatggaagga

SEQ ID NO: 14 - DNA, 20 bp, Homo sapiens
aaggtcaggttggaggagac

SEQ ID NO: 15 - DNA, 20 bp, Homo sapiens
ggcagaccgttgatccattc

SEQ ID NO: 16 - DNA, 20 bp, Homo sapiens
ggtgttgtcaagtggatcgg

SEQ ID NO: 17 - DNA, 23 bp, Homo sapiens
ggtcccccacaaagtcagaattg

SEQ ID NO: 18 - DNA, 20 bp, Homo sapiens
ggcttgtttgagagagcgag

SEQ ID NO: 19 - DNA, 20 bp, Homo sapiens
ttaacattgccccttgtgcc

SEQ ID NO: 20 - DNA, 20 bp, Homo sapiens
cagagtacacagtcgcctct

SEQ ID NO: 21 - DNA, 20 bp, Homo sapiens
gctccagtggttttctctcc

SEQ ID NO: 22 - DNA, 20 bp, Homo sapiens
tggactcaagcattcctccc

SEQ ID NO: 23 - DNA, 20 bp, Homo sapiens
agcaatctggtcaggaagct

SEQ ID NO: 24 - DNA, 20 bp, Homo sapiens
gggattgcgatgaactcagg

SEQ ID NO: 25 - DNA, 21 bp, Homo sapiens
accagcttatcttcctccacc

SEQ ID NO: 26 - DNA, 20 bp, Homo sapiens
tgcagggattacaggtgtga

SEQ ID NO: 27 - DNA, 21 bp, Homo sapiens
cccctgacattctagcttgga

SEQ ID NO: 28 - DNA, 25 bp, Homo sapiens
tgaaagatatagaggaggccaggag

SEQ ID NO: 29 - DNA, 18 bp, Homo sapiens
gtctagcccaccagcctc

SEQ ID NO: 30 - DNA, 21 bp, Homo sapiens
gggtctgagctgtacaaatgc

SEQ ID NO: 31 - DNA, 20 bp, Homo sapiens
acaccagctattcctgtggt

SEQ ID NO: 32 - DNA, 20 bp, Homo sapiens
cgcagtgtgaataagcagca

SEQ ID NO: 33 - DNA, 20 bp, Homo sapiens
ccctgatccaccactgaagt

SEQ ID NO: 34 - DNA, 20 bp, Homo sapiens
gtgggcagagatcacattcg

SEQ ID NO: 35 - DNA, 20 bp, Homo sapiens
aaacccacccttctgtcctc

SEQ ID NO: 36 - DNA, 20 bp, Homo sapiens
gctggaccctgagaatgtga

SEQ ID NO: 37 - DNA, 20 bp, Homo sapiens
ctgagagaaacgttgcggag

SEQ ID NO: 38 - DNA, 21 bp, Homo sapiens
caaaagacagagtgccttcca

SEQ ID NO: 39 - DNA, 23 bp, Homo sapiens
ctagaagccctgcctttctttgg